(19) United States
(12) Patent Application Publication
(10) Pub. No.: US 2026/0232849 A1
(43) Pub. Date: Aug. 13, 2026

(54) Substantially Monodisperse Ultrasound Responsive Nanobubble Compositions and Methods of Production and Use Thereof

(71) Applicant: University of Central Florida Research Foundation, Inc., Orlando, FL (US)

(72) Inventors: Pedram SOUTOUDEHBAGHA, Orlando, FL (US); Tara Menon PATTILACHAN, Orlando, FL (US); Mehdi RAZAVI, Orlando, FL (US)

(21) Appl. No.: 19/474,957

(22) PCT Filed: Apr. 18, 2024
(86) PCT No.: PCT/US2024/025108

§ 371 Date: Oct. 13, 2025

Related U.S. Application Data
(60) Provisional application No. 63/496,782, filed on Apr. 18, 2023.

(51) Int. Cl. A61K 49/22; A61K 9/51; A61K 31/7088; A61K 38/18; A61K 41/00; A61K 47/42

(52) U.S. Cl. CPC A61K 49/223; A61K 9/51; A61K 31/7088; A61K 38/1866; A61K 38/1875; A61K 41/0047; A61K 47/42

(57) ABSTRACT

Compositions, methods, and kits relating to substantially monodisperse, ultrasound targeted nanobubble compositions are disclosed. The nanobubble compositions comprise a hollow core containing at least one gas and a polymer shell encircling the hollow core. Methods of producing the substantially monodisperse nanobubble compositions using an ultrasound shearing-based fabrication method are disclosed. Methods of using the nanobubble compositions are also disclosed.

### CROSS REFERENCE TO RELATED APPLICATIONS/INCORPORATION BY REFERENCE STATEMENT

The subject application claims benefit under 35 USC § 119(e) of U.S. Provisional Application No. 63/496,782, filed Apr. 18, 2023. The entire contents of the above-referenced application(s) are hereby expressly incorporated herein by reference.

### STATEMENT REGARDING FEDERALLY SPONSORED RESEARCH OR DEVELOPMENT

Not Applicable.

### BACKGROUND

Osteoporosis is a systemic skeletal disorder characterized by an imbalance between bone-resorbing osteoclasts and bone-forming osteoblasts. A prominent indication is a decrease in bone quality and mass, making people more susceptible to fragility and fracture from low-energy trauma. There are approximately 10 million cases of osteoporosis in the US a year, with an additional 34 million Americans with low bone mass, putting them at a much higher risk for developing osteoporosis. It affects women more than men, where 37.7% of women and 10% of men currently suffer from osteoporosis, not including those with other underlying bone diseases such as spinal osteoarthritis. The US currently spends $10 to $17 billion a year on osteoporosis treatment, and the cost is expected to increase past $22 billion by 2030.

Parathyroid hormone (PTH) therapy is the current alternative to induce anabolic effects on bone formation, which stimulates the mechanism of osteoblasts by binding to a specific receptor and activating signaling pathways. However, various drawbacks have arisen from antiresorptive drugs like bisphosphonate, where its long-term use has been shown to induce jaw osteonecrosis and abnormal long bone fractures. Even as the most clinically used antiresorptive drug, bisphosphonate is not readily absorbed from the gastrointestinal tract, meaning that high doses are necessary, leading to greater gastrointestinal problems. These limitations center around issues of bioavailability and toxicity. Even with PTH therapy, there is possible limited efficacy on nonvertebral bone fractures and activation of bone resorption due to promotion of osteoclastogenesis resulting in chronic exposure that counteracts its anabolic purpose of promoting bone formation. As none of the current therapies for osteoporosis are without adverse effects, innovative therapies such as siRNA therapy for gene silencing are required.

Mammalian cells contain an endogenous RNA interference (RNAi) pathway that is a viable mechanism for regulating signaling pathways within cells by modulating level of gene expression. RNAi can bypass processes in the nucleus and conduct transport through the nuclear envelope. Sequence-specific small interfering RNAs (siRNA) is a portion of the RNAi complex that is able to “silence” specific gene expression having complementary gene strands that are difficult to target with conventional approaches. More particularly, siRNA therapeutic has the ability to target genes based on knowledge of the messenger RNA (mRNA) sequence. The siRNA therapeutic targets and cleaves the complementary mRNA to silence the gene.

However, the clinical translation of siRNA delivery to humans has proven challenging. The pharmacological properties of siRNA include a high anionic charge density (38-50 phosphate groups) and large molecular size (~13 kDa), both of which make siRNA ineffective in penetrating the cell membrane barrier effectively. Naked siRNA directly injected into the bloodstream or tissue are vulnerable to quick degradation and off-site targeting resulting in immune responses with Toll-like receptors. Furthermore, siRNA administered systemically requires crossing the vascular endothelial barrier before diffusing through the extracellular matrix, while avoiding kidney filtration and non-targeted cell internalization. Even after the siRNA has been up-taken into the targeted cell, they need to be released from endosomal compartments and reunite with the RNAi machinery. Additionally, siRNA must maintain resistance to nuclease degradation to properly function because of their very short half-lives of less than 6 minutes when exposed to serum nucleases. As such, there are significant challenges to using siRNA as a therapeutic and efficient methods of siRNA delivery are needed to realize its full therapeutic potential.

Therapeutic applications of gene-activated matrix, microbubbles (MBs), and nanobubbles (NBs) used as delivery systems are limited due to various disadvantages. For example, using a gene-activated matrix results in the lack of spatiotemporal control. Microbubbles suffer from various disadvantages including low stability due to high solubility of air in blood, requiring large volume for injection, and having a large micrometer size, short circulation time, low injectability, and low cellular uptake. Nanobubbles may generate undesirous reactive oxygen species and have small gas cores which provides less of a contrast agent for imaging.

A number of stimuli-responsive carrier systems currently exist for gene and/or drug delivery. Ultrasound therapy is used for internal tissues and bone healing and is considered safe and non-invasive. Low intensity pulsed ultrasound (LIPUS) and low intensity continuous ultrasound (LICUS) are two methods of ultrasound therapy. LIPUS provides mechanical energy in the form of acoustic pressure waves transmitted through tissue. The rate at which this energy is absorbed is proportional to the density of the tissue it is passing through. LICUS, when used in high intensity, can increase temperature and kill tumor cells. However, the heat produced by the ultrasound beam, in very high intensities, increases the temperature of the target site, which may interfere with the treatment.

Ultrasound therapy has previously been utilized to deliver siRNA into cells through in vitro and in vivo studies into cancer, somatic, and stem cells through the use of delivery systems such as microbubbles and nanobubbles. However, there are many unknown variables and parameters that must be determined in order to utilize ultrasound therapy as a delivery system for other applications.

Other stimuli-responsive nanosystems, such as (but not limited to) pH, reactive oxygen species (ROS), enzyme, redox, thermo, light, and carbon dioxide systems have significant drawbacks due to patient-specific circumstances as well as varying states of disease progression of the target site. For example, pH carriers can result in increased drug/gene release, but there is a large range of pH in gastrointestinal tracts well as patient-specific pH ranges, which makes it difficult to develop a nanoplatform responsive to specific pH values. Redox carriers can increase cytoplasmic release of the drug/gene but they require complex disassembly due to cleavage of thioketal linkages. Enzyme systems have high selectivity but require patient-specific enzymes and disease progression. As such, while these systems may have advantages such as high selectivity and strong correlation with disease states, known endogenous stimuli-responsive drug delivery systems have disadvantages and involve many unknown factors.

Therefore, there is a need in the art for new and improved nanobubble delivery systems, kits containing same, and methods for making and using the nanobubble delivery systems. It is to such improved systems, kits, and methods using a nanobubble platform that the present disclosure is directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 schematically depicts non-limiting embodiments of methods of producing and using substantially monodisperse nanobubble compositions in accordance with the present disclosure. Abbreviations used: AL, alendronate; NB, nanobubble(s); US, ultrasound; LIPUS, low intensity pulse ultrasound; LICUS, low intensity continuous ultrasound; PFC, perfluorocarbon.

FIG. 2 graphically depicts a tryptophan calibration curve. The tryptophan calibration curve was used to determine the total concentration of tryptophan in the samples pre- and post-LIPUS stimulation, to determine their loading and release efficacies. The curve itself was prepared through a sequence of dilutions with known concentrations, which are then analyzed and linearized into a graph with its y=mx+b formula used for tryptophan concentration calculations in the samples.

FIG. 3 contains actual representations of shearing setups constructed in accordance with the present disclosure. The representation demonstrates the combination usage of the shear rotor and the ultrasonic, pulsed sonotrode in forming NB. Shearing method 1 is set to a speed of 10,000 rpm, while the utilized (and max) speed on shearing method 2 is 2000 rpm. The simultaneously designed procedure occurs for 4 minutes total, in an on-off pulsed manner. The setup has size constraints, which require a larger beaker (i.e., 80 ml glass beaker) for both the probe and the stator of the shearing rotor to fit.

FIG. 4 contains a schematic comparison of 2 shearing device stators and mechanism of action. Panel A depicts the shearing method 2 utilizing the axial stator with mesh surrounding it, and Panel B depicts the shearing method 1 utilizing the vertical slotted head stator. Panel A was made by Pedram Sotoudehbagha. Panel B was made using Biorender.com.

FIG. 5 illustrates the ImageJ method of quantifying NB intensity across background and ROI in US. Sample phantom vessel image taken from US shearing method 1 NB imaging at 30 min post-LIPUS stimulation.

FIG. 6 graphically depicts total tryptophan release comparing the 3 NB samples of varying HSA concentrations. The time spans assessed were instantaneously after LIPUS stimulation (~0 min), 4 hours, 1 day, 3 days, and 7 days (though 7 days was excluded in graphical analysis as all the tryptophan had degraded by that timepoint in all the samples). n=3 NB productions were prepared for each concentration group, with n=2 samples from each NB production used for a total of n=6 samples at each timepoint. ns (not significant) indicates p >0.05, * indicates p≤0.05, and ** indicates p≤0.01. Statistical analysis was performed with GraphPad.

FIG. 7 graphically depicts total tryptophan release in calculating loading efficacy for the 3 NB samples of varying HSA concentrations. Only one time point was utilized (directly before LIPUS stimulation), thus a singular graph analyzing loading efficacy. n=2 NB productions were used, with n=2 replicate samples from each production, for a total of n=4 samples at each concentration. ns (not significant) indicates p >0.05. Statistical analysis was performed with GraphPad.

FIG. 8 graphically depicts total tryptophan release in calculating loading efficacy between a sheared sample (US shear method 1) and a control sample. The time spans assessed were 0 min, 4 hours, 1 day, and 3 days post-LIPUS stimulation. n=1 NB production, with n=10 samples from the NB production at each timepoint in this pilot study. ns (not significant) indicates p >0.05, * indicates p≤0.05, and **** indicates p≤0.0001. Statistical analysis was performed with GraphPad.

FIG. 9 graphically depicts PDI and effective diameters of varying NB HSA concentrations. n=3 samples for each concentration from n=1 NB production each. ns (not significant) indicates p>0.05, and * indicates p≤0.05. Statistical analysis was performed with GraphPad.

FIG. 10 graphically depicts PDI and effective diameters of control and 2 shearing fabrication methods. n=5 samples for each concentration from n=3 NB production each. ns (not significant) indicates p >0.05, ** indicates p≤0.01, and **** indicates p≤0.0001. Statistical analysis was performed with GraphPad.

FIG. 11 graphically depicts average relative intensity in NB with different HSA concentrations. Graph depiction of the effect of averaged diameter of varying HSA concentration NB on averaged relative intensity. n=3 samples for each concentration from n=1 NB production each.

FIG. 12 graphically depicts average relative intensity in NB with two different shearing methods and control groups. Graphical depiction of the effect of shearing in NB fabrication and its averaged diameter on averaged relative intensity. n=5 samples for each concentration from n=3 NB production each.

FIG. 13 contains microscope images of NB with varying HSA concentrations. The images were taken at the specified timepoints (up to 120 minutes) post-LIPUS exposure at 1 W/cm² for 4 minutes and taken using a 4× microscope. n=3 images per each 1 timepoint and NB production.

FIG. 14 contains microscope images of shearing method 1 and control NB. The images were taken at the specified timepoints (up to 120 minutes) post-LIPUS exposure at 1 W/cm² for 4 minutes and taken using a 4× microscope. n=3 images per each 1 timepoint and NB production sample.

FIG. 15 contains images of control and shearing method 1 samples pre-LIPUS exposure and stimulation. n=3 images taken from n=1 NB production. Panels A-C were individually quantified in diameter analysis.

FIG. 16 contains TEM images of shear method 1 samples and control samples. Undiluted and 10× diluted images were taken for both shear and control groups. 10× diluted for control group was excluded from analysis as NB count and resulting images were negligible. n=1 NB production for control and US shear groups, with the same production samples being diluted 10× for dilution samples.

FIG. 17 demonstrates selection of optimal sheared NB solution concentration in PBS. The images were taken directly post-LIPUS stimulation, at 0 min timepoint. n=1 sample for each concentration from n=1 US-sheared NB production.

FIG. 18 contains sheared NB samples imaged in US phantom vessel using a US scanner post-LIPUS exposure. n=5 for each timepoint. Panel A-B demonstrate the axial (A) and sagittal (B) plane views at 0 min, instantly after LIPUS exposure. Panels C-D demonstrate axial (C) and sagittal (D) views at 30 min post-LIPUS exposure, and panels E-F demonstrate axial (E) and sagittal (F) views at 60 min post-LIPUS exposure. n=5 samples from n=1 US-sheared NB production were used for image data collection.

FIG. 19 contains control NB samples imaged in US phantom vessel using a US scanner post-LIPUS exposure. n=5 for each timepoint. Panel A-B demonstrate the axial (A) and sagittal (B) plane views at 0 min, instantly after LIPUS exposure. Panels C-D demonstrate axial (C) and sagittal (D) views at 30 min post-LIPUS exposure, and panels E-F demonstrate axial (E) and sagittal (F) views at 60 min post-LIPUS exposure. n=5 samples from n=1 control NB production were used for image data collection.

FIG. 20 contains an in vivo comparison of livers between control and NB injected mice. n=1 sample from n=1 NB production for each mice imaged.

FIG. 21 contains US image from 40 mg concentration of US shearing method 1 at 0 min. The NB were imaged instantaneously at moment of injection. n=1 sample image from n=1 NB production.

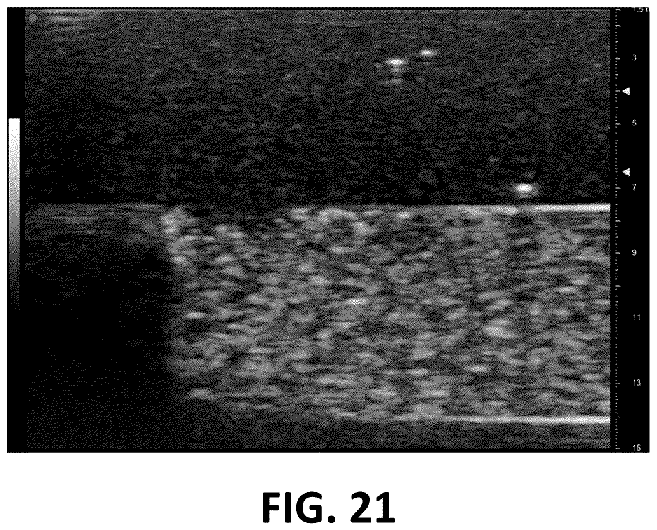

### DETAILED DESCRIPTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting in any way.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” and “approximately” are used to indicate that a value includes the inherent variation of error for a composition/apparatus/device, the method being employed to determine the value, or the variation that exists among the study subjects. That is, the terms “about” and “approximately” and variations thereof are intended to include not only the exact value qualified by the term, but to also include some slight deviations therefrom, such as deviations caused by measuring error, manufacturing tolerances, wear and tear on components or structures, settling or precipitation of cells or particles out of suspension or solution, chemical or biological degradation of solutions over time, stress exerted on structures, and combinations thereof, for example. In particular, for example, but not by way of limitation, when the term “about” is utilized, the designated value may vary by plus or minus ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a composition, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “patient” includes human and veterinary subjects. In certain embodiments, a patient is a mammal. In certain other embodiments, the patient is a human, including, but not limited to, infants, toddlers, children, young adults, adults, and elderly human populations. “Mammal” for purposes of treatment refers to any animal classified as a mammal, including human, domestic and farm animals, nonhuman primates, and zoo, sports, or pet animals, such as dogs, horses, cats, cows, etc.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, reversing, alleviating, inhibiting the progress of, preventing, or reducing the occurrence of at least one condition, disease, or disorder, or one or more symptoms thereof. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/disorder in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concepts, the present disclosure relates to compositions(s), system(s), and kit(s), as well as methods for making and using same, that are based on a substantially monodisperse ultrasound targeted nanobubble system (such as, but not limited to, an ultrasound targeted nanobubble destruction (UTND) system) synthesized using (for example, but not by way of limitation) an in situ sonochemical shearing-based fabrication method. The substantially monodisperse nanobubble system can be used for various theranostic applications, including (but not limited to) as an ultrasound contrast agent and/or as a gene, drug, and/or oxygen delivery system for various conditions, diseases, and disorders, including (but not limited to) various bone conditions, diseases, and disorders, various types of cancer, and various neurodegenerative conditions, diseases, and disorders.

Certain non-limiting embodiments of the present disclosure are directed to a substantially monodisperse ultrasound responsive targeted nanobubble composition. Each nanobubble comprises a hollow core containing at least one gas; a polymer shell encircling the hollow core; at least one therapeutic agent disposed in the hollow core and/or encapsulated within the shell; and at least one targeting agent incorporated in the polymer shell and/or attached to its surface. The nanobubble composition is substantially monodisperse in that the composition has a polydispersity index value in a range of from about 0.0 to about 0.20.

Any gas known in the art for use as a contrast agent for ultrasonography may be utilized as the gas present in the hollow core of the nanobubble composition in accordance with the present disclosure. In certain particular (but non-limiting) embodiments, the gas present in the hollow core is a perfluorocarbon (PFC) gas. Non-limiting examples of PFC gases that can be utilized include perfluoropentane (PHP, C₅F₁₂) and perfluorohexane (PFH, C₆F₁₄).

The shell of the nanobubble composition may be formed of any polymer known in the art or otherwise contemplated herein that is useful in forming a polymer shell for ultrasonography reagents. Non-limiting examples of polymers that may be utilized include albumin, polyethylene glycol (PEG), poly lactic acid (PLA), poly lactic-co-glycolic acid (PLGA), chitosan, gelatin, and the like, as well as combinations and co-polymers thereof.

In a particular (but non-limiting) embodiment, the polymer shell comprises albumin and/or PEG.

Any therapeutic agents known in the art or otherwise contemplated herein that would benefit from targeted delivery via an ultrasound responsive nanobubble system may be utilized in accordance with the present disclosure. The at least one therapeutic agent may be selected for treatment of various diseases, disorders, or conditions, including (but not limited to) bone conditions, diseases, and disorders (such as, but not limited to, fractures, bone defects, osteoporosis, osteoarthritis, and bone cancer) as well as neurodegenerative diseases (such as, but not limited to, Alzheimer's disease, Parkinson's disease, and amyotrophic lateral sclerosis (ALS)) diseases) and various types of cancer.

In particular (but not by way of limitation), the at least one therapeutic agent may be a gene or other nucleotide-based agent (such as, but not limited to, an siRNA or microRNA), a protein, a drug, and/or a gas (such as, but not limited to, oxygen).

Non-limiting examples of therapeutic agents that may be utilized in accordance with the present disclosure include an siRNA; a microRNA; a gene sequence; an antioxidant; a peptide or protein; CRISPR/CS9 delivery; nanoparticles; extracellular nanovesicles (i.e., exosomes); and drugs; as well as any combinations and/or conjugations thereof. Non-limiting examples of siRNAs that may be utilized in accordance with the present disclosure include Cathepsin K (CTSK) siRNA, VEGF siRNA (for the inhibition of angiogenesis in tumors), histone deacetylase 5 (HDAC5) siRNA, osteoprotegerin (OPG) siRNA, LDL receptor-related protein 5 (LRP5) siRNA, sclerostin (SOST) siRNA, Receptor activator of nuclear factor kappa-B ligand (RANKL) siRNA, Noggin siRNA, pyruvate kinase M2 (PKM2) siRNA, doublecortin like kinase 1 (DCLK1) siRNA, multi-siRNA, and other types of siRNAs for (for example, but not by way of limitation) musculoskeletal disorders and cancer therapies. Non-limiting examples of gene sequences that may be utilized in accordance with the present disclosure include regenerative genes such as vascular endothelial growth factor (VEGF), a bone morphogenetic protein (BMP, such as but not limited to BMP2 or BMP7), hepatocyte growth factor (HGF), osteocalcin, and the like. Non-limiting examples of antioxidants that may be utilized in accordance with the present disclosure include vitamins, such as, but not limited to, vitamin C, D, and/or E. Non-limiting examples of proteins and peptides that may be utilized in accordance with the present disclosure include VEGF, a BMP (such as, but not limited to, BMP2 or BMP7), HGF, and the like, as well as any peptide fragments thereof. Non-limiting examples of nanoparticles that may be utilized in accordance with the present disclosure include cerium oxide, mesoporous bioactive glass, bioceramics, ion doped glass-ceramics, and the like. Non-limiting examples of particular combinations of therapeutic agents that may be encapsulated together within the nanobubble compositions of the present disclosure include a combination of two siRNAs, a siRNA/miRNA combination, a siRNA/gene sequence combination, a siRNA/protein or peptide combination, a siRNA/antioxidant combination, a siRNA/CRISPR/CS9 delivery combination, a siRNA/nanoparticle combination, a siRNA/extracellular nanovesicle combination, a siRNA/drug combination, and the like.

In particular (but non-limiting) embodiments, the therapeutic agent is CTSK siRNA. CTSK siRNA can be used to silence genes, such as the Cathepsin K gene which causes osteoporosis. In other particular (but non-limiting) embodiments, the therapeutic agent includes another siRNA for treatment of a bone condition, disease, or disorder.

Alternatively (and/or in addition thereto), the oxygenated nanobubble compositions of the present disclosure can also be used as oxygen nano shuttles to deliver oxygen to the bone or cancer, prevent tumor hypoxia, and improve responsivity to chemo, radiation, or photodynamic therapy. Oxygen delivery can also help with tissue regeneration and wound healing.

Alternatively (and/or in addition thereto), the at least one therapeutic agent may include one or more drugs and/or growth factors for targeted cancer theranostics. Regenerative genes such as (but not limited to) vascular endothelial growth factor (VEGF), bone morphogenetic proteins (BMP), hepatocyte growth factor (HGF), and osteocalcin can be incorporated into the nanobubbles for promoting angiogenesis and tissue regeneration.

In certain particular (but non-limiting) embodiments, the nanobubble composition may include two or more therapeutic agents for codelivery of the two or more therapeutic agents to the same target. When multiple therapeutic agents are present, the agents may be of the same or different classes of molecules. For example (but not by way of limitation), the nanobubble composition may include siRNA and microRNA; siRNA and gene sequence; siRNA and antioxidant; siRNA and protein; etc.

In a particular (but non-limiting) embodiment, the nanobubble composition comprises at least two therapeutic agents that include CTSK siRNA and at least one gene sequence selected from VEGF and a BMP.

The nanobubble composition is functionalized by incorporation within the polymer shell (and/or attachment to the surface of the polymer shell) of one or more targeting agents to hone the nanobubble composition to specific cells/targets at the site or localization of a condition, disorder, or disease to be treated. The targeting agent(s) may be any protein, peptide, or compound capable of honing the nanobubble composition to specific cells/targets at the site or localization of a condition, disorder, or disease to be treated. In some non-limiting embodiments, the at least one targeting agent targets the nanobubble composition to at least a portion of a musculoskeletal system of a subject/patient. For example, but not by way of limitation, the targeting agent can be alendronate, a molecule which serves as a bone-targeting biomarker to hone to osteoclasts. In addition, the nanobubble composition can be functionalized with one or more targeting agent(s) that target to the bone cells or other cell types (for example, but not by way of limitation, at least one of liver, pancreas, neurological, or cancer cells).

In one particular (but non-limiting) embodiment, the at least one gas in the nanobubble composition is perfluorocarbon gas, the polymer shell comprises albumin and/or PEG, and the at least one targeting agent comprises alendronate and/or at least one bone targeting peptide.

In particular (but non-limiting) embodiments, the nanobubble composition may further comprise one or more imaging agents or biomarkers encapsulated inside the nanobubble (i.e., disposed in the hollow core of the nanobubble) and/or incorporated in the polymer shell of the nanobubble in order for the nanobubble to be imageable via various imaging techniques, such as (but not limited to) fluorescent imaging, Raman imaging, and magnetic resonance imaging (MRI). The nanobubbles can also be used (with or without imaging agent) as a contrast agent for contrast-enhanced ultrasound (CEUS) imaging.

The nanobubble compositions of the present disclosure may be provided with any size that allows the compositions to function as described herein. In particular, the nanobubble compositions should be provided with a size that allows the nanobubble compositions to facilitate cell internalization, easily leak from the vasculature (which can be an advantage for tumor therapy), and/or sequentially release multiple therapeutic agents. Nanobubbles (NBs) are capable of crossing the endothelial gap in blood vessels and enter, aggregate, and target the interstitial space of cells (including tumor interstitial spaces). The ability of the NB to penetrate into the vasculature and remain after intravenous injection places a high NB concentration due to accumulation in the target site, increasing the permeability of the cell after ultrasound exposure.

For example, but not by way of limitation, the nanobubble composition may be provided with a diameter of about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, and the like, as well as any value that falls between two of the above values (i.e., about 52 nm, about 77 nm, etc.); the nanobubble composition may also have a diameter in a range formed of two of the above values (i.e., a range of from about 20 nm to about 200 nm, a range of from about 20 nm to about 150 nm, a range of from about 15 nm to about 100 nm, a range of from 15 nm to about 50 nm, etc.), as well as a range formed of two values, each of which falls between two of the above values (i.e., a range of from about 22 nm to about 164 nm, a range of from about 47 nm to about 143 nm, etc.).

The substantially monodisperse nature of the nanobubble compositions of the present disclosure facilitates more programmable and personalized use of the nanobubbles as a platform for drug and gene delivery systems, as well as for ultrasound contrast agent applications. Most importantly, increased monodispersity improves dose precision and reproducibility of the drug/gene delivery, which are ideal for clinical and medical utility. The nanobubble compositions of the present disclosure may be provided with any polydispersity index (PDI) value that allows the compositions to function as described herein. Non-limiting examples of PDI values for the nanobubble compositions of the present disclosure include about 0.0, about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, and the like, as well as a range formed of two of the above values (i.e., a range of from about 0.0 to about 0.20, a range of from about 0.1 to about 0.20, etc.).

Certain non-limiting embodiments of the present disclosure are directed to a system or library that comprises at least one of any of the substantially monodisperse nanobubble compositions disclosed or otherwise contemplated herein. For example, but not by way of limitation, the system may include two, three, four, five, six, seven, eight, nine, ten, or more different nanobubble compositions. The different nanobubble compositions may contain the same or different therapeutic agents; for example, the presence of different therapeutic agents in the nanobubble compositions would allow for use in a concurrent therapy treatment plan, and/or the different therapeutic agents may act synergistically with one another. When two or more nanobubble compositions are present in the system, the two or more nanobubble compositions may be designed for administration simultaneously or wholly or partially sequentially.

In particular (but non-limiting) embodiments, the library/system comprises a plurality of substantially monodisperse nanobubble compositions that each have a different size. For example, but not by way of limitation, the library/system may include one or more of a nanobubble composition having a size of about 20 nm in diameter, a nanobubble composition having a size of about 25 nm in diameter, a nanobubble composition having a size of about 50 nm in diameter, a nanobubble composition having a size of about 75 nm in diameter, a nanobubble composition having a size of about 100 nm in diameter, a nanobubble composition having a size of about 125 nm in diameter, a nanobubble composition having a size of about 150 nm in diameter, a nanobubble composition having a size of about 175 nm in diameter, and/or a nanobubble composition having a size of about 200 nm in diameter.

Certain non-limiting embodiments of the present disclosure are directed to methods of using any of the libraries/systems disclosed or otherwise contemplated herein. For example (but not by way of limitation), sequential therapeutic agent release can be obtained via administration of a nanobubble library of various sizes; administration of the library can result in prolonged silencing of key genes in osteoporotic bones, thereby reducing the dosing schedule.

Certain non-limiting embodiments of the present disclosure are directed to a method of preparing any of the substantially monodisperse, ultrasound responsive targeted nanobubble compositions disclosed or otherwise contemplated herein. The method comprises the steps of: (1) mixing at least one therapeutic agent with at least one gas and at least one polymer to form a first mixture; (2) sonicating and shearing the first mixture to form nanobubbles that comprise a polymer shell formed about a hollow core containing the at least one therapeutic agent and the at least one gas; (3) isolating the nanobubbles; and (4) contacting the nanobubbles with a targeting agent to form a second mixture and incubating the second mixture under conditions that allow the targeting agent to be incorporated in the polymer shell of the nanobubbles.

The sonicating and shearing actions are performed simultaneously, and each of these actions may be performed using any apparatus known in the art or otherwise described herein. For example, but not by way of limitation, the sonication is performed by a sonotrode, and the shearing is performed by a rotor-stator system.

Each of the method steps may be performed at any temperature and for any period of time that allows for the formation of the nanobubble compositions. In certain particular (but non-limiting) embodiments, at least steps (2) and/or (4) can be performed at a temperature in a range of from about 0° C. to about 4° C., and step (4) can be performed for a period in a range of from about 24 hours to about 48 hours.

It will be understood that steps (1), (2), (3), and (4) can be performed substantially simultaneously or wholly or partially sequentially. For example, but not by way of limitation, siRNA, polymers, proteins, targeting agents, and/or drugs, and the like can be incorporated into the nanobubble shell via step-by-step injection during the ultrasonic shearing step to generate layer-by-layer nanobubble assemblies without the need for a second process to functionalize nanobubbles. Therefore, the use of ordinal terminology is for purposes of illustration only and should not be construed as limiting to the order or sequence of steps performed.

In addition, the process parameters can be optimized to achieve the best monodispersity and desired nanobubble size. Non-limiting examples of parameters that can be optimized include duty cycle, ultrasound intensity, shearing rate, time, and/or frequency.

The nanobubbles can be isolated in step (3) by any methods known in the art. In a particular (but non-limiting) embodiment, step (3) comprises a centrifugation step. Alternatively, the nanobubble size isolation can be performed using a nanofiltration approach.

When the nanobubble composition includes at least one imaging agent, the at least one imaging agent may be added to step (1) and/or to step (4) so that the at least one imaging agent is disposed in the hollow core and/or incorporated in the polymer shell.

Certain non-limiting embodiments of the present disclosure are directed to substantially monodisperse nanobubble compositions produced by any of the methods described or otherwise contemplated herein.

Certain non-limiting embodiments of the present disclosure are directed to a method that comprises the step of (1) administering an effective amount of at least one of any of the substantially monodisperse, ultrasound responsive targeted nanobubble compositions disclosed or otherwise contemplated herein to a patient in need thereof.

In certain particular (but non-limiting) embodiments, the method may further comprise a step of exposing the patient to ultrasound for in vivo diagnostic imaging of a portion of the patient.

In certain particular (but non-limiting) embodiments, the method may further comprise the steps of: (2) allowing the nanobubble composition to travel through the patient so that the targeting agent binds to a target within the patient; and (3) exposing the patient to ultrasound. The ultrasound application may be for diagnostic imaging purposes; alternatively (and/or in addition thereto), the ultrasound may emit acoustic waves based on a determined frequency with an intensity and duration to rupture the nanobubble composition at the target within the patient.

In certain particular (but non-limiting) embodiments, the method may comprise the step of exposing the patient to ultrasound prior to performing step (1).

In certain particular (but non-limiting) embodiments, the method may include performing steps (1), (2), and (3) above, in combination with step (4): administering an effective amount of a second ultrasound responsive targeted nanobubble composition to a patient in need thereof, wherein the nanobubble composition comprises a hollow core containing at least one gas, a polymer shell encircling the hollow core, at least one therapeutic agent disposed in the hollow core and/or encapsulated within the shell, and at least one targeting agent incorporated in the polymer shell and/or attached to its surface, wherein the at least one targeting agent targets the nanobubble composition to at least a portion of a musculoskeletal system of the patient.

In a particular (but non-limiting) embodiment, the method is further defined as a method of treating a bone condition, disease, or disorder in a patient. For example, but not by way of limitation, the bone condition may be osteoporosis, osteoarthritis, a bone fracture, a bone defect, cancer, and the like.

In another particular (but non-limiting) embodiment, the method is further defined as a method of treating or reducing the occurrence of cancer in a patient.

In another particular (but non-limiting) embodiment, the method is further defined as a method of treating or reducing the occurrence of a neurological disease, disorder, or condition in a patient. Non-limiting examples of neurological diseases, disorders, and conditions treatable by the methods of the present disclosure include Alzheimer's disease, Parkinson's disease, and amyotrophic lateral sclerosis (ALS).

In some non-limiting embodiments, the ultrasound can be selected from low intensity pulsed ultrasound (LIPUS), low intensity continuous ultrasound (LICUS), or focused ultrasound (FUS).

In addition to the use of pulsed or continuous waveforms, the methods of the present disclosure can also utilize any output intensities, frequencies, exposure times, and post-exposure times that provide for expansion and rupture of the nanobubble compositions and delivery of the at least one therapeutic agent to the target. For example (but not by way of limitation), the method may utilize an output intensity in a range of from about 1 W/cm² to about 3 W/cm², a frequency in a range of from about 0.5 MHz to about 1.5 MHz, an exposure time in a range of from about 1 minute to about 20 minutes, and a post-exposure time in a range of from about 1 minute to about 7 days. In certain non-limiting embodiments, the LICUS, LIPUS, or FUS can be connected to an imaging probe such as ultrasound, for image-guided diagnostic purposes as well as nanobubble destruction and therapeutic delivery.

Through application of ultrasound (probe), the nanobubble grows and expands until it ruptures and delivers its contents into a target cell. The system functions by controlled sequential release, where the ultrasound parameters can be optimized (e.g., exposure time, intensity, frequency, pulsed/continuous waveform, etc.) to customize to the severity of the disease (e.g., low intensity to superficial fractures versus high intensity for deep lesions). Sonoporation and cavitation can be detected within the nanobubble after low-frequency ultrasound is applied. In one non-limiting embodiment, the ultrasound parameters are 3 W/cm² output intensity, 1 MHz frequency, 5 min exposure time, and continuous waveform. However, it will be understood that any ultrasound parameters known in the art or otherwise contemplated herein may be utilized in accordance with the methods of the present disclosure.

Certain non-limiting embodiments of the present disclosure include a kit for preparing an ultrasound responsive targeted nanobubble composition and/or for providing treatment using an ultrasound responsive targeted nanobubble composition. The kit may include one or more of any of the substantially monodisperse nanobubble compositions disclosed or otherwise contemplated herein. Alternatively, the kit may comprise the various different components of any of the substantially monodisperse nanobubble compositions disclosed or otherwise contemplated herein. The kit may further include one or more additional components, depending on the consumer.

For example, if the kit is being used in a research lab, the kit may comprise (i) a therapeutic ultrasound probe, and (ii) a substantially monodisperse nanobubble composition that does not contain a therapeutic agent and/or a polymer. An exemplary research lab kit may also comprise (i) a therapeutic ultrasound probe, and (ii) a substantially monodisperse, ultrasound responsive targeted nanobubble composition comprising at least one therapeutic agent disposed in a hollow core of the composition. An exemplary research lab kit may also comprise (i) a therapeutic ultrasound probe, and (ii) a substantially monodisperse, ultrasound responsive targeted nanobubble composition comprising at least one therapeutic agent disposed in a hollow core of the composition, and further comprising a polymer shell encircling the hollow core, and a targeting agent incorporated in the polymer shell. An exemplary research lab kit may also comprise (i) a therapeutic ultrasound probe, and (ii) a substantially monodisperse, ultrasound responsive targeted nanobubble composition comprising a polymer shell encircling the hollow core and a targeting agent incorporated in the polymer shell.

An exemplary research lab kit may also comprise (i) a therapeutic ultrasound probe, and (ii) a substantially monodisperse, ultrasound responsive targeted nanobubble composition comprising CTSK siRNA disposed in a hollow core of the composition. An exemplary research lab kit may also comprise (i) a therapeutic ultrasound probe, and (ii) a substantially monodisperse, ultrasound responsive targeted nanobubble composition comprising CTSK siRNA disposed in a hollow core of the composition, and further comprising a polymer shell encircling the hollow core and alendronate incorporated in the polymer shell. An exemplary research lab kit may also comprise (i) a therapeutic ultrasound probe, and (ii) a substantially monodisperse, ultrasound responsive targeted nanobubble composition comprising a polymer shell encircling the hollow core and alendronate incorporated in the polymer.

If the kit is being used for medical purposes for local delivery, such as in a hospital, where the location already has a therapeutic ultrasound probe, the kit may comprise a substantially monodisperse, ultrasound responsive targeted nanobubble composition comprising at least one therapeutic agent disposed in a hollow core of the composition. An exemplary medical kit may also comprise a substantially monodisperse, ultrasound responsive targeted nanobubble composition comprising at least one therapeutic agent disposed in a hollow core of the composition, and further comprising a polymer shell encircling the hollow core and a targeting agent incorporated in the polymer shell. An exemplary medical kit may also comprise a substantially monodisperse, ultrasound responsive targeted nanobubble composition comprising siRNA disposed in a hollow core of the composition. An exemplary medical kit may also comprise a substantially monodisperse, ultrasound responsive targeted nanobubble composition comprising siRNA disposed in a hollow core of the composition, and further comprising a polymer shell encircling the hollow core and alendronate incorporated in the polymer shell.

The kit can further include a set of written or pictorial instructions (or information on how to obtain instructions, either written or pictorial, from the internet) explaining how to use the kit. A kit of this nature can be used in any of the methods described or otherwise contemplated herein.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

In this Example, a novel method for synthesizing nanobubbles using ultrasonic shearing is disclosed, which has clinical utility on drug and gene delivery systems. The nanobubbles are composed of a perfluorocarbon gas core and an albumin outer shell, which are stabilized with human serum albumin (HSA). The method includes optimization phases to determine the optimal amount of HSA for stabilizing the nanobubbles and to evaluate two different ultrasonic shear methods for their effects on the monodispersity, protein release rates, and nanobubble sensitivity to ultrasound. In addition to serving as a platform for drug/gene delivery systems, the nanobubbles have applications as an ultrasound contrast agent, which is useful in biodistribution studies and clinical diagnostics. As described in this Example, the nanobubble composition has been tested in a phantom vessel and in a pilot study in vivo, providing support for theranostic applications of the nanobubbles for use in various conditions, diseases, and disorders, including osteoporosis and the bone. Also disclosed is the formulation of a nanobubble library with different sizes formed through a personally programmed ultrasonic shearing method, thus having a wide range of use-cases as a result.

The novel features of the nanobubbles fabrication method include the use of ultrasonic shearing in the synthesis of nanobubbles and the optimization of the nanobubble composition and synthesis for clinical applications. In addition, these features can specifically be combined with the bone-healing low intensity pulse ultrasound (LIPUS) of 1 to 2 W/cm². This method of ultrasonic shearing simultaneously combines the use of a sonotrode for ultrasonication with a rotor-stator system for homogenization of the perfluorocarbon (PFC) emulsion. This technique prevents the perfluorocarbon (PFC)-in-water separation, decreases sound wave absorption, and produces monodisperse, size-tunable nanobubbles. The utilization of LIPUS has been demonstrated to effectively stimulate the growth and subsequent rupture of nanobubbles through a process known as ultrasound-mediated nanobubble destruction.

This Example is directed to a novel ultrasonic (US) shearing-based fabrication method for synthesizing nanobubbles, which can then be utilized as a platform for any theranostic applications in clinical medicine, such as (but not limited to) image guided drug/gene delivery systems. This in situ sonochemical shearing synthesis of nanobubbles incorporates a perfluorocarbon gas core (300 μl) and an albumin outer shell, which are then incorporated into phosphate buffered saline (4 ml) and later sonicated with an US probe while simultaneously shearing with a rotor-stator system. The initial optimization phase (which utilized ultrasonication in the absence of shearing) included experimenting with various amounts of human serum albumin (HSA), which stabilizes the nanobubble gas core. Of the parameters examined (20 mg, 40 mg, and 80 mg HSA), 40 mg HSA significantly outperformed (p<0.05) the other parameters on tryptophan protein release on several time points, such as after 1 day and 3 days. Additionally, the monodispersity of the nanobubbles was another metric that was optimized, as it gives a more programmable nature to the nanobubbles as a platform for clinical usage and in personalized medicine. The 40 mg HSA samples once again demonstrated lowered average polydispersity values (0.194 as opposed to 0.309 in 20 mg HSA and 0.289 in 80 mg HSA samples). In the second optimization procedure, 2 ultrasonic shear methods were evaluated in comparison to the control, with one method utilizing a vertical slotted head stator (shearing method 1), and the other utilizing an axial stator with mesh (shearing method 2). In these methods, US sonication was simultaneously performed alongside the shearing procedure. Shearing can reduce the effect of ultrasonic attenuation in PFC emulsion and prevent PFC-in-water separation. The second optimization procedure demonstrated that in a set of similar assays and experiments, shearing method 1 was more optimal in its high monodispersity, protein release rates, and ultrasound-responsiveness of nanobubbles in comparison to the control. The final phase of this project assessed the ultrasound contrast agent properties and intensity values of nanobubbles in a phantom vessel overtime, and also included a pilot study in vivo imaging nanobubbles in the liver. The project continues to optimize and expand on the theranostic applications of the US sheared nanobubbles in vivo and ex vivo in osteoporosis and the bone.

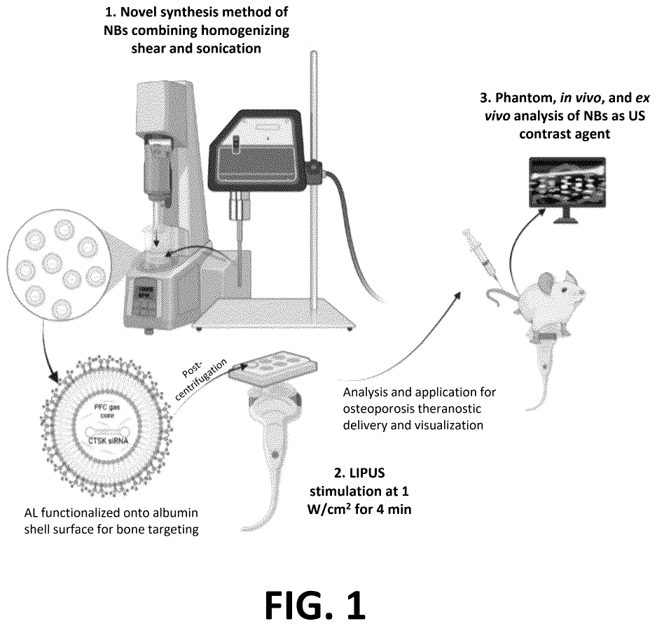

A diagram of the prototypical ultrasonic shearing apparatus that was tested is shown in FIG. 1.

The ultrasonic shearing fabrication method for nanobubble synthesis disclosed herein is unique and novel over the prior art. Ultrasound stimulation has been used for several microbubble studies across the years, as microbubbles have widely been understood to have echogenic, responsive cores that can facilitate drug delivery and function as an ultrasound contrast agent (as seen, for example, in Batchelor et al. (2020) ACS Applied Materials and Interfaces, 12(26):29085-29093). The ability of nanobubbles to produce echoes in ultrasound imaging has not been thoroughly examined because of their tiny gas core. Despite this, the Example demonstrated that the specific, substantially monodisperse nanobubbles produced herein can be visualized using ultrasound in laboratory settings. Lastly, while low intensity pulsed ultrasound (LIPUS) is a known mechanism for US-mediated nanobubble destruction, its utility in the context of drug/gene delivery is rarely studied, despite its applications in bone fracture healing and ease in implementation (Zhang et al. (2020) Stem Cells Int, 2020:8863577). In this Example, a distinctive implementation of ultrasonic shearing was utilized in conjunction with LIPUS for not only its bone healing properties, but also multimodally for US contrast imaging, gene/drug delivery, and targeted release.

International Patent Application Publication No. WO 2022/212446 (published Oct. 6, 2022, and referred to herein after as “the ‘446’ application;” and for which the entire contents are hereby expressly incorporated herein by reference) discloses nanobubble compositions and methods of production and use thereof. The '446 application utilizes a different method of synthesis to create the nanobubbles, which involves the mixing of a therapeutic agent with PFC and a polymer, followed by sonication and isolation of the nanobubbles. In contrast, the methods of the present disclosure involve a novel ultrasonic shearing-based synthesis method, which has been optimized for use in theranostic applications in clinical medicine such as drug and gene delivery systems. The optimization process described in this Example includes the evaluation of different amounts of HSA for stabilizing the nanobubbles, as well as the testing of two different ultrasonic shear methods (vertical slotted head stator setup and axial stator setup) for their effects on monodispersity, protein release rates, and nanobubble sensitivity. In addition, the '446 application was limited to just ~4 ml of solution and did not utilize shearing in combination with ultrasound sonication. In contrast, the methods of the present disclosure combine both ultrasound sonication and shearing, enabling the production of nanobubbles in a much larger volume (up to ~40 ml of solution). This is a significant improvement, as it makes the production of nanobubbles more efficient and cost-effective for scaling up and commercialization. For production of larger volumes of nanobubbles, multi-ultrasonic shearing can be used, and scaling up is achieved using multiple sonotrodes and rotor-stators integrated horizontally and/or vertically.

Jadhav et al. (Industrial & Engineering Chemistry Research (2021) 60(23):8597-8606) describe a method for generating bulk nanobubbles using a high-shear rotor-stator device. In contrast, the present disclosure utilizes a combined ultrasonic shearing approach to synthesize nanobubbles. The methods of the present disclosure allow for production of substantially monodisperse nanobubbles in large volumes, and these nanobubbles have unique therapeutic applications as they are specially optimized to work (for example, but not by way of limitation) alongside LIPUS at 1 W/cm², which promotes bone healing and has shown stimulation in bone growth/osteogenesis. In contrast, the bulk nanobubbles described to have formed in Jadhav et al. are less effective as a gene/drug delivery system, as they are highly stable and have a long half-life, thus leading to less sensitivity to LIPUS, poorer drug release rates, reduced uptake by cells, and potential toxic effects on cells and tissues from circulating in the blood for an extended period of time.

On the other hand, the methods of the present disclosure have demonstrated the ability to release proteins at higher volumes and at higher rates, which can reflect its more effective ability to release drugs and genes. The substantially monodisperse nanobubbles constructed in accordance with the present disclosure can also function as a US contrast agent. Overall, the methods of the present disclosure provide a unique and innovative approach to nanobubble synthesis and mass production for commercialization in the medical industry which provides improved synthesis efficiency, multimodal and therapeutic applications, and demonstrated effectiveness as an US contrast agent.

The present disclosure offers several advantages over currently available technology. One of the main advantages is the improved monodispersity values of the nanobubbles synthesized using the ultrasonic shearing-based method. The optimized synthesis method resulted in more monodisperse nanobubbles with a polydispersity index (PDI) of less than 0.200, which facilitates more programmable and personalized use of the nanobubbles as a platform for drug and gene delivery systems, as well as for ultrasound contrast agent applications. In addition, increased monodispersity improves dose precision and reproducibility of the drug/gene delivery, which are ideal for clinical and medical utility.

In addition to the improved monodispersity, the ultrasonic shearing-based synthesis method enables the production of nanobubbles in larger volumes, approximately 40 ml of solution, as well as larger volumes. By utilizing a larger rotor-stator and sonotrode, production of large volumes of nanobubbles is made possible. This increased production capacity allows for the synthesis of nanobubbles to be more cost-effective and efficient and thus makes it more widely accessible to patients. The nanobubbles synthesized using the ultrasonic shearing-based method also release protein at higher volumes and rates (as shown in the Examples), which reflects its more effective ability to release drugs and genes.

The ultrasonic shearing-based synthesis method also demonstrates use in medical diagnostics, as the nanobubbles can function as an ultrasound contrast agent in phantom vessel imaging studies.

Ultrasonic sheared samples (n=5 replicates for both groups) displayed higher US contrast intensity compared to nanobubble samples fabricated with ultrasonication alone (i.e., produced by the methods disclosed in the '446 application), with a maximum region of interest (ROI) intensity of 60.624 in the former compared to a maximum ROI intensity of 36.71 in the latter. In addition, the nanobubbles are specially optimized to work alongside low intensity pulsed ultrasound (LIPUS), at 1 W/cm², which promotes bone healing and has shown stimulation of bone growth and osteogenesis. The ultrasonic sheared nanobubbles presented a higher responsiveness to LIPUS as compared to nanobubbles fabricated with ultrasonication alone in our study. At a proportionally higher volume, the samples fabricated with ultrasonication alone (i.e., produced by the methods disclosed in the '446 application) only expanded up to a maximum average of 108 μm (n=50 NBs), while the ultrasonication-shearing samples expanded up to a maximum average of 754 μm (n=50 NBs) prior to destruction and release. Thus, the combination of LIPUS and the optimized ultrasonic-sheared nanobubbles has a synergistic effect, as the LIPUS not only promotes osteogenesis but also induces the acoustic induction and eventual release of the therapeutic drug to the region of interest. This has practical applications in the treatment of bone diseases such as (but not limited to) osteoporosis, as well as in targeting tumors or cancers in other parts of the body.

As such, the ultrasonic shearing-based synthesis methods of the present disclosure demonstrated superior performance in terms of protein release rates and nanobubble sensitivity to ultrasound compared to the non-sheared control (i.e., nanobubbles prepared by the methods of the '446 application).

The shearing device used in the synthesis of the nanobubbles can be adjusted to prepare specific sizes, and various meshes around the stator, various pore/opening sizes in the stator, and/or various rotor designs can be incorporated to adjust the size of the nanobubbles as well. The ultrasonic shearing process can be controlled by ultrasound intensity, duty cycle, duration, and shearing rate to create nanobubbles with desired size and monodispersity. This personalized approach to nanobubble synthesis can be applied in various fields, including (but not limited to) water treatment and clinical medicine. In the field of clinical medicine, the ability to synthesize nanobubbles with specific sizes and characteristics may be useful in gene therapy and delivery to a region of interest, as well as in medical diagnostics as a contrast agent. This personalized approach to nanobubble synthesis is particularly important for incorporating personalized medicine in gene therapy, as it permits the production of nanobubbles tailored to the specific needs of the patient.

In addition to the clinical medicine applications, the ability to synthesize nanobubbles with specific sizes and characteristics also has practical applications in other fields beyond medicine, such as (but not limited to) in wastewater management to remove organics pollutants like grease, and to disinfect water. The versatility of the ultrasonic shearing-based synthesis method allows for the production of nanobubbles with specific sizes and characteristics suitable for a wide range of use-cases. Furthermore, the ultrasonic shearing method advances the sonochemistry field in micro/nanoparticle synthesis.

As such, the ultrasonic shearing-based synthesis methods of the present disclosure overcome the limitations of currently available technology in terms of the versatility and personalized nature of nanobubble synthesis. The ability to adjust the shearing prototype device and method and incorporate various meshes/pores within the stator, rotor design, or control the process parameters such as ultrasound intensity, duty cycle, shearing rate, and time permits production of nanobubbles with specific sizes and characteristics, and these nanobubbles with specific sizes and characteristics have the ability to be applied in a wide range of use-cases, including in clinical medicine and wastewater management. This personalized approach to nanobubble synthesis is particularly important for incorporating personalized medicine in gene therapy, as it enables production of nanobubbles tailored to the specific needs of the patient and as a multiplex drug/gene delivery approach for sequential release.

Thus, the ultrasonic shearing-based nanobubble fabrication methods of the present disclosure provide a novel approach for large-scale production of nanobubbles with clinical applications, such as (but not limited to) drug and gene delivery systems and medical diagnostics. The improved monodispersity of the US-sheared nanobubbles allows for more consistent and programmable use in these systems. Additionally, the methods of the present disclosure allow for the production of nanobubbles in larger volumes, making it more efficient and cost-effective. The methods of the present disclosure address the need for large-scale manufacturing of size-controlled nanobubbles with higher monodispersity in the commercial industry. The nanobubbles synthesized using the ultrasonic shearing-based method can also be used as an ultrasound contrast agent in medical diagnostics. The nanobubbles produced in Example 1 demonstrated the ability to act as a contrast agent over time within a phantom vessel, indicating their utility in biodistribution studies and clinical diagnostics.

### Example 2

This Example proposes a novel, ultrasonic shearing-based fabrication approach in combination with an improved NB composition and synthesis for mass production of NB in osteoporosis treatment and as a US contrast agent, though not limited to this sole clinical application. The present disclosure improves upon predecessor NB studies by also increasing their total monodispersity, loading, and release efficacy, while also opting for a lower US intensity. Bulk NB can hold industrial applications, such as in treating organic pollution in wastewater, as they improve flotation efficiency for the removal of pollutants such as grease, and their collapse induces the formation of free radicals for the oxidation and disinfection of toxins and pathogens. However, their longevity and unresponsiveness to US frequencies for expansion and collapse make it undesirable for gene and drug delivery purposes. The present Example is directed to producing NB for gene and drug delivery purposes using the disclosed US shearing-based fabrication approach.

### Materials and Methods

### 1. In Situ Sonochemical Synthesis of Nanobubbles

The in situ sonochemical synthesis of NB was prepared by incorporating it into a combination of perfluorocarbon (PFC), phosphate buffered saline (PBS; Gibco), and human serum albumin (HSA; Sigma Aldrich). The standard default concentrations involve 300 μl PFC, 4 mL PBS, and 40 mg of HAS; however, in this project, CTSK siRNA was not involved or incorporated yet until the optimal NB parameters have been finalized. This step involved testing different amounts of HSA (e.g., 20 mg, 40 mg, 80 mg). During this process, the mixture remained mostly translucent in color. Following completion of this mixing process, the mixture was then sonicated using a US probe (Fisherbrand™ Model 120 Sonic Dismembrator) while in an ice bath for 2.5 minutes, in an on-and-off pulse manner. Once the solution had visibly changed color from translucent colorless to white, the samples were transferred to 100 ml centrifuge tubes, washed out using deionized water, and were finally balanced. Once the samples were all balanced in mass, they were ultracentrifuged (Optima XPN-100 Ultracentrifuge, Beckman Coulter) at 15,000 rpm for 30 minutes at 22° C. Following centrifugation, the samples should form a circular or oval pellet at the bottom, which is then easily accessible after cutting the tube and pouring out the remaining supernatant. Though the NB are ready to be used for US stimulation and imaging right away, if storage is desired, the samples can be sealed airtight properly to prevent any gas or liquid leakage. Finally, the samples were placed in the rear end of the fridge to prevent any deformations, as well as prevent heat and energy exchange with the environment as much as possible.

### 2. Acoustic Induction of Nanobubble Growth and Expansion Using LIPUS

Acoustic induction occurred during the actual US stimulation process, in which a US therapy device and probe (New Pocket Sonovit Portable Ultrasonic Therapy Device—45 mm probe) were utilized. Portions of the NB pellets from synthesis were taken (roughly 20 mg) and transferred into 2 mL of PBS, which was then transferred to 6-well plates. Once the US device was turned on, and Aquasonic Clear® Ultrasound Gel was applied thoroughly underneath the plate, the probe was placed underneath, and the modality was selected on the device screen (i.e., LIPUS program with 1 W/cm² intensity, 1 MHz frequency, for 4 minutes in a pulsed manner). Immediately following the completion of US stimulation, real-time growth, and expansion of the NB can be monitored through optical imaging microscopy (AmScope 40×-2000× Biological Research Microscope with 5.1MP Camera). Videos and images may be taken throughout a certain timespan to determine timestamps of interest, such as the NB moment of rupture. These images can then be organized in time-lapse sequential order to visually depict the growth, expansion, and rupture of the NB. Additionally, the images can serve as a tool to further analyze the size parameters of individual bubbles (i.e., diameter and total area) and approximate the rate of change of these parameters over time. This was achieved by utilizing ImageJ, manually counting, and quantifying the average of 100 NB for each time point. As NB are spherical in morphology, spherical overlays were casted on the NB to calculate area, which was used to calculate the diameter from the area of a circle formula (A=πr2). The selection of NB was chosen at random and was limited to 100 for consistency and to provide a sizeable data selection.

### 3. Selection of Final Parameters for Quantitative and Qualitative Analysis

This general method of preparation of NB was standardized and used for further comparisons and experiments, though finalizing on the method itself underwent a process of trial and error. For example, introducing vortexing prior to sonicating was a withdrawn mechanism in preparation, as it resulted in no changes or improvements and increased complexity. Additionally, we assessed various quantities of albumin, from 5 mg to 500 mg HSA, to observe any significant effects on NB capabilities and effectiveness. Ultimately, it was determined that <20 mg HSA led to minimal, if any, formation of NB even under high intensity of US (2-3 W/cm²), while a large quantity of HSA (>80 mg) was detrimental to release due to the high amount of albumin serving as a stabilizer. With this similar reasoning and questions on the total effect on NB, various US intensities ranging from 0.08 to 3 W/cm² were studied in combination with the range of varying HSA amounts stated previously. Low intensities <1 W/cm², such as that of 0.08 W/cm², showed minimal to no NB formation during all time points, when visualized under the microscope. Intensities ≥2 W/cm² were also unfavored in the process of optimizing NB as high-intensity US has been found to lead to adverse side effects in humans such as skin burn and leg pain. Additionally, we had evaluated 3 W/cm² intensity in continuous US, which resulted in limb dysfunction in mice; this could be attributed to muscle, vessel, or nerve damage due to the high US intensity or heat formed from the application. Thus, various compositions of 20, 40, and 80 mg HSA was paired with 1 W/cm² LIPUS was selected for our first objective of optimizing NB for theranostic applications in drug/gene delivery systems, for osteoporosis and potentially other diseases and regions in the human body.

### 4. Assessment of the Loading and Release Efficacy of Nanobubbles Using Tryptophan

Though the general imaging of NB post-LIPUS stimulation in a microscope provides some information on effective parameters, a crucial parameter of the NB to analyze includes the loading (uptake) and release efficacy, as these NB serve as theranostic drug delivery systems and optimally would necessitate high loading efficacy at earlier time points, and high release efficacy at later time points. L-tryptophan (Sigma Aldrich) was a cost-effective protein to use for measuring these parameters as tryptophan, due to its conjugated pi bonds, can be quantified using ultraviolet (UV) fluorescence measurements on the SpectraMax iD5 (Molecular Devices) multi-mode microplate reader and spectrophotometer. It was selected initially instead of CTSK siRNA as it offers advantages such as low cost and easy availability. Tryptophan would serve as an initial test protein that measures the effects of NB formulation on drug loading-release kinetics.

To initially introduce tryptophan into the NB itself, the synthesis procedure would have to be partly affected, though the overall sequence of steps would remain the same. Rather, we first had to calculate the optimal amount of tryptophan to prepare prior to the microplate reader measurements. To achieve this, however, a tryptophan dilution series and standard calibration curve must be prepared first. This is due to tryptophan having a limit of detection (LOD), a measure of its sensitivity, which we measured to be 2.16 μM. To prepare our dilution series and standard calibration curve, we prepared a sequence of micropipette tubes, prepared a high molar concentration of 100 mM tryptophan in PBS buffer, and continuously prepared dilutions of 50 mM, 25 mM, 10 mM, 5 mM, 2.5 mM, 1 mM, 500 μM, 250 μM, 100 μM, 50 μM, 25 μM, and 0 (PBS blank). The dilutions were placed into n=5 microplate wells in each column and analyzed by the SpectraMax iD5 spectrophotometer at a UV reading between 280 and 360 nm wavelengths. The optical density values were then annotated and graphically depicted through Excel, with the linear portion of the curve being up to 250 μM, our reference point of interest to determine the total amount of tryptophan loaded and released in the NB (R²=0.99). The R² value, also known as the correlation of determination, measures the percent of the dependent variable's total variation that is gauged by the independent variable, with an R² of 1.0 indicating a perfect linear model and relationship. Accordingly, the tryptophan concentration we opted to use was 16 mg/ml, although we had experimented with higher and lower values which yielded insignificant results out of the dilution curve range.

The actual preparation and synthesis of NB with tryptophan incorporated differs at the beginning, as tryptophan is temperature-sensitive (as are all amino acids) and also light-sensitive, which could induce degradation. Additionally, through repeated trial-and-error, we found the tryptophan would not dissolve easily into 4 ml PBS. Thus, the preparation must be conscious of these limitations and attempt to counter them. We finalized on a procedure in which the tryptophan concentration of 16 mg/ml was multiplied by 4 to 64 mg/4 ml PBS, and then placed in a room temperature (~25° C.) sonicating water bath, to dissolve as much tryptophan into the PBS solution as possible without degrading it. 300 μl PFC and the respective amount of albumin was added to the tube afterwards, particularly as PFC is volatile and may escape the solvent during the mild heating procedure. Lastly, a foil wrap was always kept around the tubes throughout the entire synthesis procedure to prevent light-caused degradation of tryptophan. Following this initial diverging step, the remainder of the steps—namely, sonicating, balancing for centrifugation, and the rotor parameters for centrifugation remain the same. Another diverging step occurs at the last step, when preparing the tubes and typically isolating the pellets. Instead, the supernatant was extracted and placed into a separate tube, as it can be useful in comparing loading efficacies between samples by looking at how much tryptophan is in the supernatant and its optical density values. This supernatant can be read on the microplate and was diluted on the orders of 1:20, 1:100, and so on to get optical density values within the linear range of the tryptophan calibration curve formed. The dilution factor, in addition to the amount of deionized water added during elution for centrifugation and balancing, must be kept in mind as this total dilution amount was multiplied into the final optical density to yield the true concentration of tryptophan. Calculating release efficacy requires 20 mg of the pellet to be put into 2 ml of PBS, which was then stimulated under 4 minutes of LIPUS at 1 W/cm². The reading of the release efficacy values occurred at several different time-points to give insight to amount of release overtime, such as instantaneous (t=0), 4 hours, 1 day, and 3 days. Previously, 7 days was also measured, but it was found that nearly all tryptophan was degraded by this time point and was not comparable. This procedure was performed on both the initial parameterization experiments with 20, 40, and 80 mg HSA NB, and was also assessed in a pilot study comparing the sheared sample and the 40 mg HSA control sample.

### 5. Dynamic Light Scattering for Determining the Polydispersity of Nanobubbles

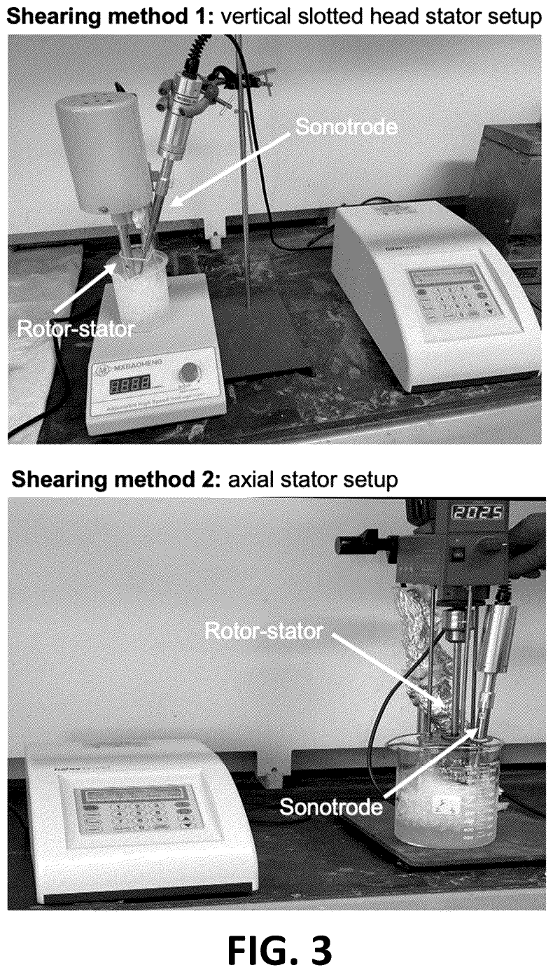

Dynamic light scattering (DLS) was used to aid in calculating both the polydispersity index (PDI) and the effective diameter of the NB. In the initial comparisons of 20, 40, and 80 mg albumin concentrations, 1 NB was synthesized with triplicates for each. For the final comparisons of finding the ideal NB parameters, 3 different NB were synthesized for the 40 mg control, US shearing method 1, and US shearing method 2 (FIG. 3). It was then separated into 5 replicates each. To prepare for DLS, first 20 mg of the prepared pellet must be dispersed into 2 ml PBS within a 2 ml microcentrifuge tube. For DLS, the formed solute must be diluted approximately 100 times, using triple-filtered water, preventing multiscattering. To prepare this water, deionized water was transferred over 3 times to another bottle by using a syringe with a filter. We performed these dilutions by first forming 1:10 dilutions and finally, 1:100. The final amount in the 1:100 labeled tubes was transferred to a borosilicate glass tube (Fisherbrand 12×75 mm) and would then be taken to the DLS machine (Brookhaven Instruments) for further analysis.

The DLS instrument was initially set up and prepared by turning on the laser, rotating the goniometer from 10° to 90°, and ensuring that the environment is at room temperature (25° C.). The glass samples were wiped down with acetone, loaded into the instrument, and the filter was changed from closed (C) to 640 nm. The laser shutter is slid open, and the laser is connected to the application interface. The experiment was run for approximately 2 minutes, with values such as effective diameter, PDI, intensity, and a correlation function forming over time with populated data points. After each run, the experiment was saved, the laser shutter was closed alongside with the filter, and the glass tube was once again wiped down with acetone. This procedure was repeated as much as necessary, with the datasets compared against each other and used to generate graphs.

6. Shearing with US Sonication as a Novel Synthesis Method

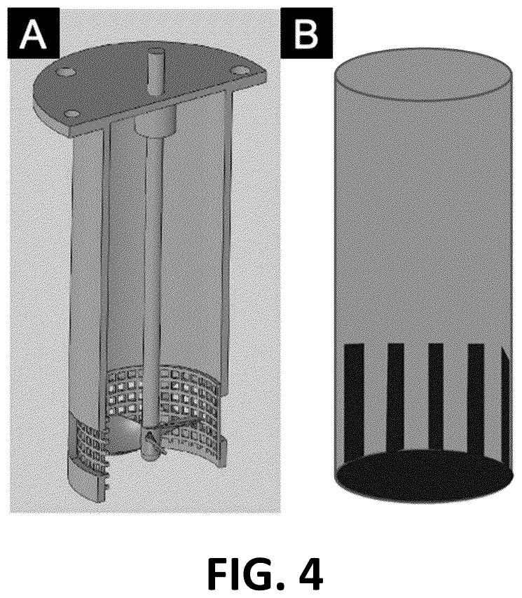

Following the determination of the optimal concentration from the 20, 40, and 80 HSA concentrations, we wanted to further see if any other parameters could aid in the reduction of effective diameter and PDI. We chose to incorporate shearing into our synthesis procedure, as a novel method that would improve upon those parameters in addition to loading and release efficacy. US shearing would require rotors to be placed into the liquid, and with its dimensions, it would require a larger volume than the previously used ~4.3 ml. Instead, all parameters were then multiplied ×10, to allow for the rotor to go in while maintaining the same concentration (i.e., 4 ml PBS, 40 mg HSA, 300 μl PFC would become 40 ml PBS, 400 mg HSA, and 3 ml PFC instead to an approximate total volume of ~43 ml). Thus, instead of tubes, the traditional synthesis steps would occur in 50 ml beakers. The shearing would be introduced during the 2.5 minute on-off sonication, being manually turned on and off in tune with the sonicator. 2 different shearing methods were assessed—US shearing method 2 with an axial stator (spun at ~2000 rpm), and US shearing method 1 with a vertical slotted head stator (spun at ~10,000 rpm). The latter of the 2 is categorized as US shearing method 1, with the former labeled as US shearing method 2 (FIGS. 3-4).

Prior to shearing, a two-step cleaning process occurred in which it was wiped thoroughly, sonicated for 10 minutes in 50 ml of 70% ethanol in the US bath, and following another round of thorough wiping, again placed for 10 minutes in 50 ml deionized water inside of the bath. During shearing, the rotor and mesh (FIG. 4) was spun in deionized water and wiped with 70% ethanol following each usage, to remove any contaminants and residue from previous trials. Following shearing and sonication, the steps remain the same in respect to balancing, centrifuging, and isolating the pellet.

### 7. Transmission Electron Microscopy (TEM) for Characterization

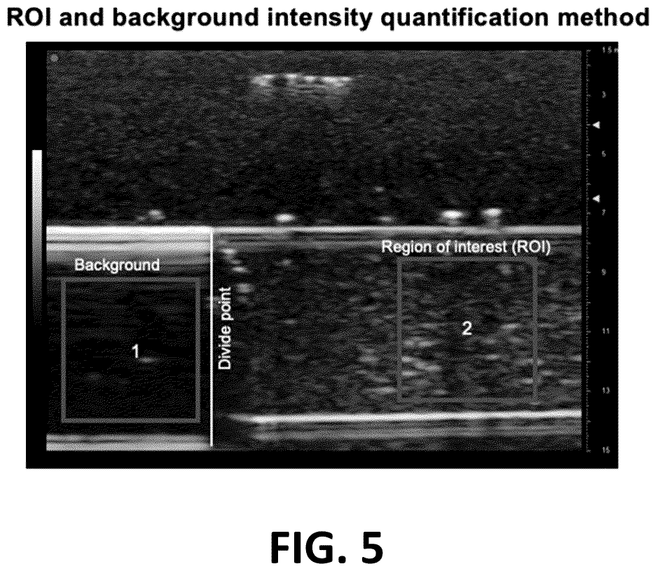

Transmission electron microscopy (TEM; Jeol JEM-1011) was performed on the sheared and control samples, to observe the fine, ultra-structural details and size parameters at higher magnification. Approximately 2.5 μl of NB solution (20 mg per 2 ml PBS), undiluted and diluted 1:10 in triple-filtered water, was transferred to a copper grid (FCF-3 400-CU, Electron Microscopy Sciences-EMS, Hatfield, PA, USA) and was analyzed at UCF Materials Characterization Facility. Images obtained were further calibrated and scaled using ImageJ (FIG. 5). TEM shines an electron beam through the NB specimen, passing through several condenser lenses and apertures before reaching the main screen with the sample plated on the grid. The white/light regions in TEM images are sections of the sample that are less dense as more electrons had transmitted through, whereas black/dark regions are, conversely, denser regions as less electrons had passed through.

8. US Imaging NB with Phantom Vessel

US imaging was performed in a phantom vessel (Your Design Medical, Ultrasound Guided IV Intravenous Trainer Phantom) using the Vevo 3100 Preclinical Imaging System US scanner to visualize the NB (n=5 for control and sheared samples each) over a 1-hour period post-LIPUS. Measurements were taken post-initial 1 W/cm² LIPUS stimulation (t=0 min, 30 min, and 1 hour) to track visualization and expansion of NB as a contrast agent over time. Quantification of NB contrast and brightness was done by utilizing ImageJ intensity measurements across a background and a region of interest (ROI) square overlay, which was applied evenly across the US images. The intensity scale and values were measured from 0 (black) to 256 (brightest white) using the 8-bit color system. Signal to noise ratio was also accounted for by dividing the ROI by the background, thus accounting for the strength of the intensity relative to background noise.

### 9. Guide for In Vivo and Ex Vivo Experiments and US Imaging

In vivo experiments in mice were conducted in which mice were injected into the tail vein with 40 mg non-sheared NB that were functionalized with varying concentrations of AL in a series of 24-hour experiments. The mice were also shaved in both front and back abdominal regions, which may improve imaging resolution and be a region for US imaging. X-ray images were taken of the mice first (n=3 each of male and female mice) and the mice were sacrificed ~24 hours later, with their organs (femur, tibia, liver, kidneys, spleen, heart, and a lower portion of the spine) transferred into 6-well plates and imaged in the X-ray machine. Live imaging of functionalized NB in vivo using the US machine and transducer was attempted, with images of specific regions (i.e., femur, tibia, and liver) collected, compared, and analyzed between control and injected mice groups.

### 10. Statistical Analysis Methods for Quantitative Data

One-way ANOVA with post-hoc Tukey HSD Tests were performed on individual sets of samples using GraphPad (in both initial HSA parameterization and shear/control comparisons). Experimental group sets were organized into n=3 NB production replicates for initial pilot HSA parameterization studies in microscopic imaging, protein loading/release studies, as well as DLS comparisons. The HSA parameterization experiments did not proceed to US imaging in the phantom vessel. To be more precise, the sample size was later increased to n=5 NB production replicates for US shearing method 1/US shearing method 2/control comparisons in DLS comparisons and n=5 replicate samples from n=2 NB productions. US imaging in the phantom vessel, however the microscopic image study remained at n=3 NB productions for each group. Additionally, only 1 group each (n=1 from n=1 NB production) was used for the pilot protein loading/release study in control vs. US shearing and requires further replicates. GraphPad was also used for performing unpaired t-tests for 2 group comparisons (as seen in control vs. US-sheared groups), and for creating and outlining statistical significance in graphs.

In analyzing quantitative data, ns (not significant) indicates a p-value >0.05. Statistical significance was assigned when p≤0.05 (*), p≤0.01 with symbol **, p≤0.001 with symbol ***, and p≤0.0001 with symbol ****. Graph results were displayed in mean and standard deviation (SD).

### Results

### 1. Tryptophan Loading and Release Efficacy

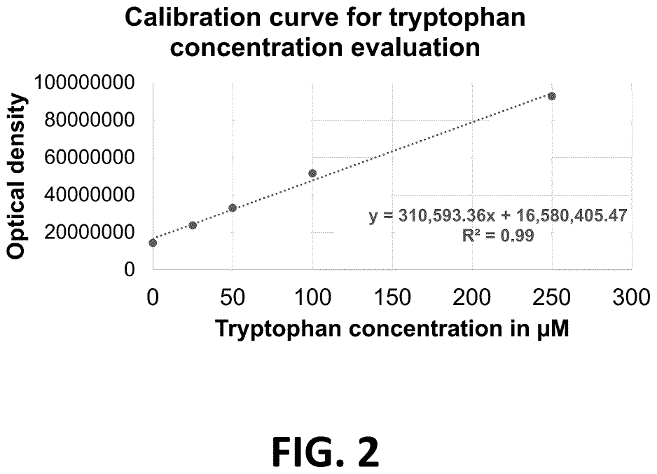

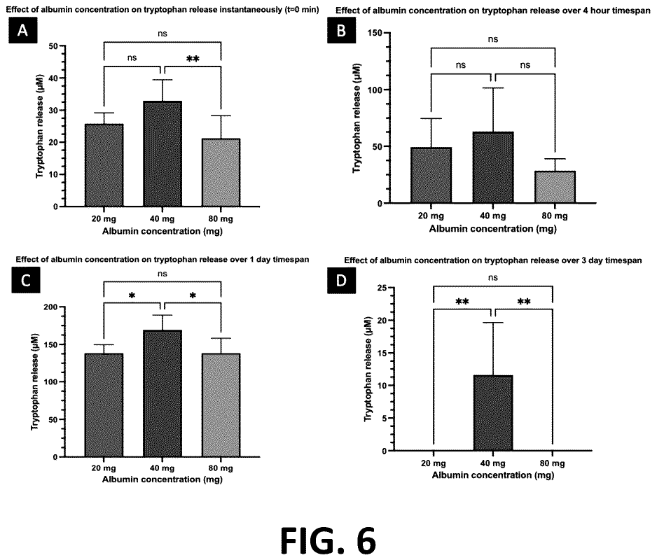

Using the SpectraMax iD5 spectrophotometer and the tryptophan calibration curve prepared through known dilutions (seen in FIG. 2), the optical densities of the samples post-LIPUS were received. In the first test of protein loading and release efficacy, the parameters were between 20, 40 and 80 mg HSA, which would form the shell for the NB. The samples were analyzed over the span of 7 days, with checkpoint times being 0 min (directly after LIPUS stimulation), 4 hours, 1 day, 3 day, and 7 days. The original amount of tryptophan loading during synthesis was 80 mM, determined from initially preparing the NB with 64 mg Trp per 4 ml of solution, thus, the loading efficacy of the NB with 20 mg of HSA was 84.2±0.4%, for 40 mg it was 83.8±0.5%, and for 80 mg, it was 82.3±2.4%. The averaged tryptophan release values from a set of n=3 samples for each selected concentration of HSA NB, with 2 from each sample for a total of 6 wells each read for spectrophotometry analysis, are shown in FIG. 6. Instantly after LIPUS (0 min), the 20 mg HSA NB released 26.21 μM of tryptophan; 40 mg HSA NB released 32.9 μM of tryptophan, and the 80 mg HSA NB released 20.1 μM of tryptophan. After 4 hours, the 20 mg HSA NB had released 55.4 μM of tryptophan; 40 mg HSA NB released 54.5 μM of tryptophan, and the 80 mg HSA NB released 26.7 μM of tryptophan. After approximately 24 hours (1 day), the samples released average tryptophan concentrations of 135.4 μM (20 mg HSA), 168.6 μM (40 mg HSA), and 133.3 μM (80 mg HSA). After 3 days, only the 40 mg HSA NB released tryptophan at all, releasing 10.4 μM.

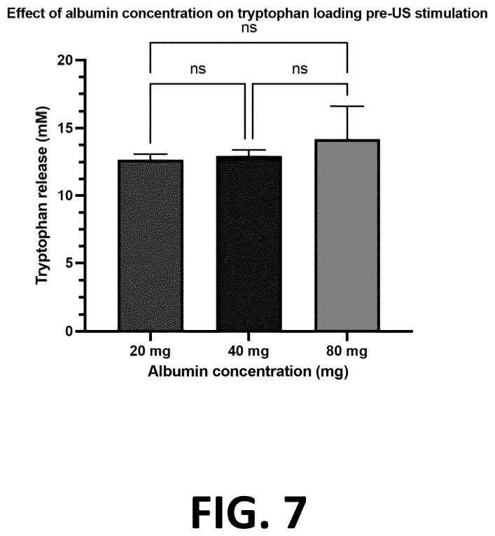

Prior to LIPUS stimulation, the overall loading efficacy of Trp was also assessed for n=4 samples each (FIG. 7). No significant differences were discernable between the 3 different HSA (mg) samples, as well as in the shear methods vs. control group experiments. In analyzing the loading efficacy, higher tryptophan release is undesirable.

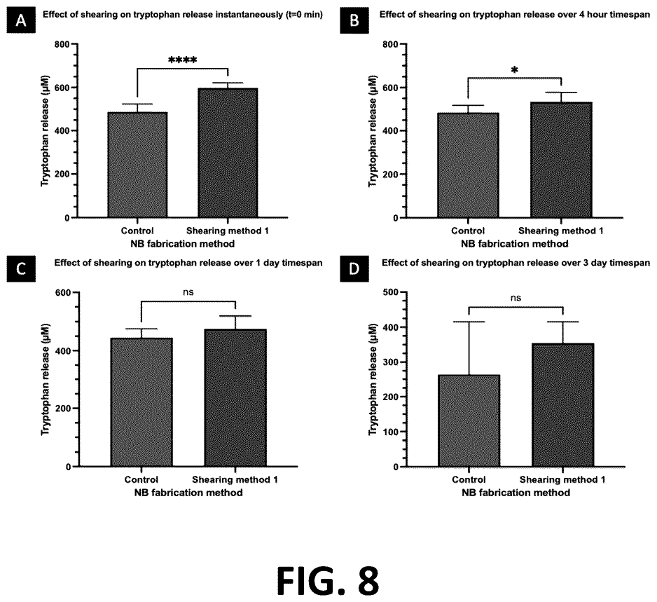

After the protein loading/release experiments of FIGS. 6-7 and subsequent quantification studies through DLS and manual image analysis, the selected NB HSA was 40 mg, and this would proceed as the control group in addition to 2 other shear method comparisons. In a pilot n=1 NB production study, Trp loading, and release efficacies were analyzed between a non-shear control and shearing method 1 (vertical slotted head stator). Like the previous Trp experiments and analysis of loading efficacies, there was no significant difference between release values in between the control and shearing method 1; thus, no graph was produced. The sheared NB samples released approximately 0.6 mM, with a 99.2% loading efficacy, while the control had released 0.67 mM, with a 99.1% loading efficacy. However, a graph was produced for the Trp release efficacy over the same timespans (excluding 7 days' time point to account for Trp degradation): 0 min, 4 hours, 1 day, and 3 days (FIG. 8). The total loaded Trp concentration at the start is the same as the previous experiments (at 80 mM) as the overall concentration was maintained despite the change in synthesis procedure. During the synthesis procedure, 40 ml solution was used in preparing the three variables and 64 mg of Trp was added per 4 ml of solution (total 640 mg). Yet, the amount of tryptophan released was significantly higher for both the control and shearing method 1 compared to values seen in the HSA concentration NB comparisons. At 0 mins post-LIPUS, shearing released 597.9 μM of Trp, and the non-shear control released 488.0 μM of Trp. After 4 hours, the sheared sample released 534.5 μM, and the control released 484.9 μM; after 1 day the sheared released 475.2 μM and control released 445.2 μM; and after 3 days the sheared sample released 354.3 μM and the control released 264.01 μM.

### 2. Determination of PDI and Effective Diameter Through Dynamic Light Scattering

DLS analysis was performed on both comparison studies: the 20 vs. 40 vs. 80 mg HSA concentration samples, as well as the control vs. shear method 1 (vertical slotted head stator) vs. shear method 2 (axial stator). Through the DLS software (Brookhaven Instruments), data for sample PDI and effective diameter averages and intensity graphs were constructed.

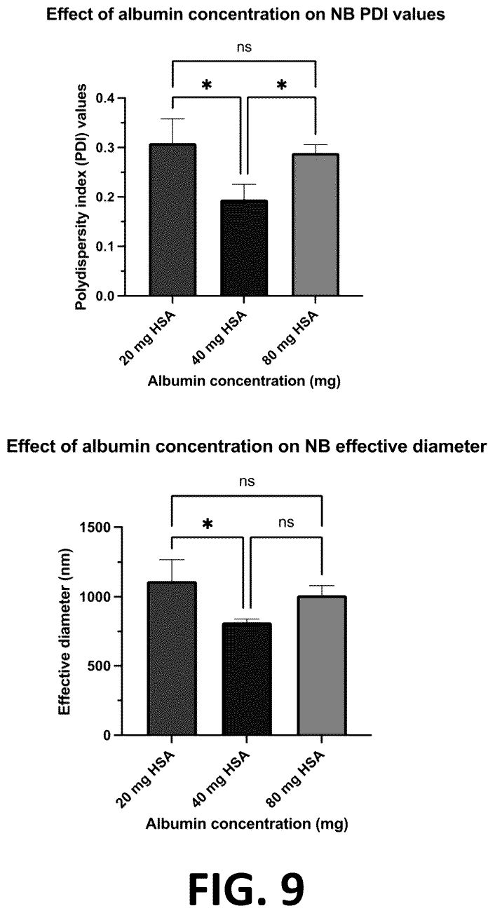

As shown in FIG. 9, the results demonstrate that NB PDI values were significantly lower at a 40 mg HSA concentration as opposed to 20 mg or 80 mg HSA, though the two are not statistically significantly different between themselves. The same conclusion was drawn for NB effective diameter, except that 40 mg and 80 mg HSA concentrations were not significantly different.

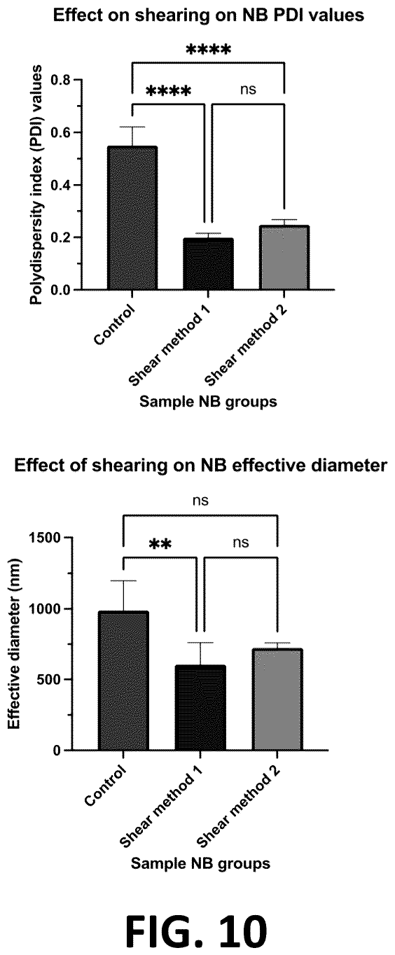

The results comparing shearing methods and the control group (FIG. 10, top panel) demonstrated that NB PDI values are significantly reduced in both shear methods compared to the control, and that the two shearing methods are not significantly different. The NB effective diameter graph (FIG. 10, lower panel) results demonstrate that shear method 1 is preferable to control in the reduction of effective diameter, though shear method 2 is not significantly different to either control or shear method 1.

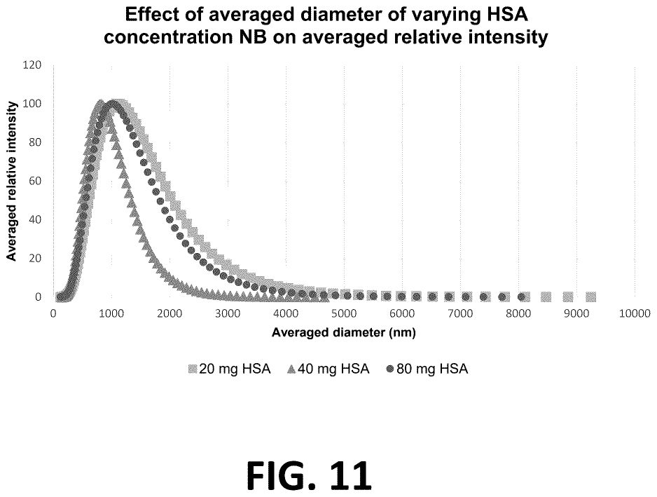

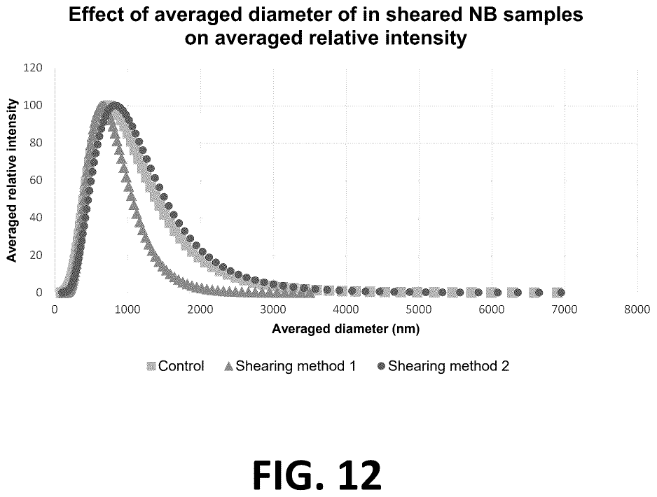

The average relative intensity distribution visualizes the amount of light that is scattered by the NB particles in the solution, with the peak roughly correlating with the averaged diameter, as the larger and more apparent NB would contribute more to the light scattering. The graphs of FIGS. 11-12 demonstrate that the 40 mg HSA sample has a distinctly lower averaged diameter at the peak of averaged relative intensity, with comparable results for shearing method 1 in the shear methods vs. control groups.

It is worth nothing that effective diameter results from DLS is different than the TEM characterization and manually quantified effective diameter values, with DLS showing larger diameters for the NB than TEM. This is due to DLS assessing the hydrodynamic size of NB, thus adding to its hydrodynamic diameter. Additionally, DLS overestimates the average effective diameter as it frequently selects for the larger NB (and thus larger effective diameter) that scatters light more strongly compared to the smaller NB.

### 3. Characterization of NB Samples Post-LIPUS on Microscope

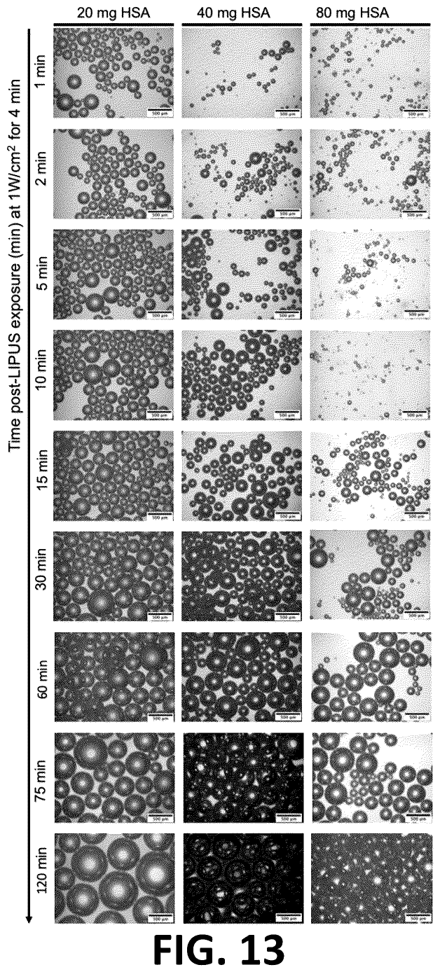

After the DLS and protein loading/release studies, it was determined that shearing method 1 with the vertical slotted head stator (also with 40 mg HSA in shell composition) would proceed as the final selected shearing option for comparison with the 40 mg HSA control. NB were initially characterized for 2 hours through the 4× lens on the AmScope 40×-2000× Biological Research Microscope, prior to TEM imaging, and the results are shown in FIG. 13. In the 20, 40, and 80 mg HSA NB comparisons, all 3 sample NB had expanded and grew over time, with measurements at 1 min post-LIPUS (for n=50 counted for each group/timepoint) demonstrating diameters of 178±30 μm (20 mg), 99±25 μm (40 mg), and 73±30 μm (80 mg). By 30 min, diameters of the NB had expanded to 296±30 μm, 276±30 μm, and 189±30 μm respectively, and finally at 120 min, was at 628±40 μm, 531±25 μm, and 331±30 μm.

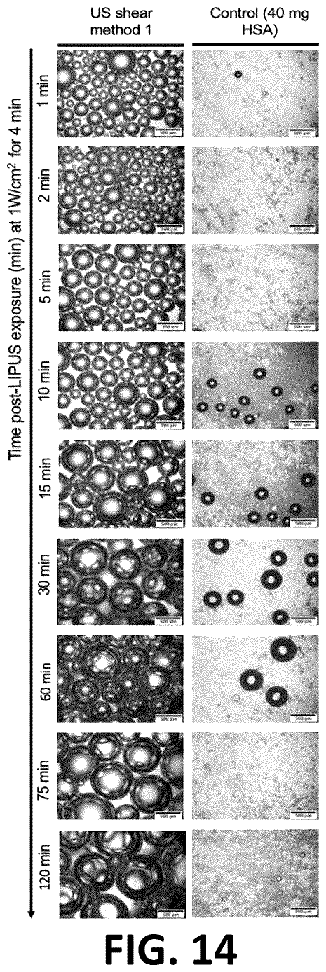

For the second comparison between the shearing method 1 and control samples (n=3 and 2 NB productions respectively), the procedure was identically performed and displayed more NB growth (diameter, size) as well as overall monodispersity and homogeneity in the sheared sample, rather than the control, which had some growth and expansion in certain NB but retained an innumerable number of smaller NB over the entire 2-hour time span. At 1 min post-LIPUS (n=50 counted for each group/timepoint), diameters were 42±35 μm (control) and 237±55 μm (US sheared). By 30 min, diameters expanded to 108±35 μm is the control, likely due to several outlier-like large NB included in the quantification. In contrast, at 30 min, the diameter of US sheared NB was 337±60 μm. By the final 120 min timepoint, larger NB was not found in the control NB sample, resulting in a low diameter at 31±30 μm while the diameter for the US sheared NB sample was 754±60 μm. It is important to note that the images for 40 mg HSA in FIGS. 13 and 40 mg HSA control in FIG. 14 appear different as the setup in the US-sheared and control comparisons is different in terms of volume of the PFC-in-water emulsion. Groups in FIG. 13 had ~4.3 ml while Groups in FIG. 14 had ~43 ml.

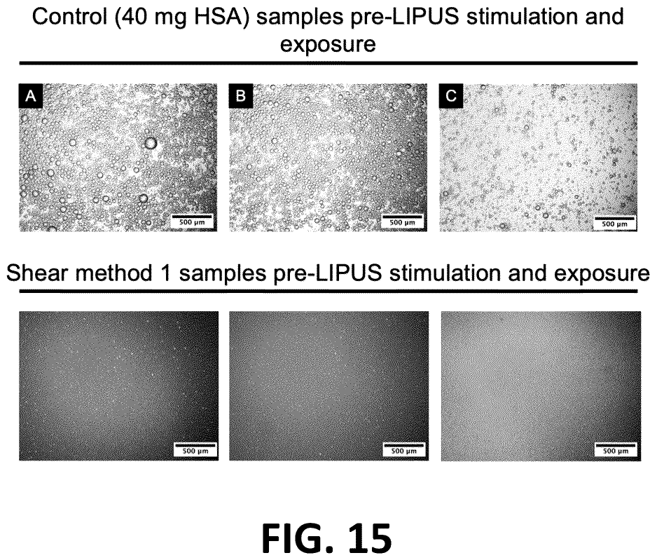

To control for the effect of LIPUS, prior to stimulating, 1 sample each (n=1) of the shear method 1 and control sample had images taken under the microscope to account for the NB in the solution prior to growth, expansion, and eventual destruction. Majority of the NB would expand in the shear method 1 samples, and many of the bulk, smaller NB in the control would not expand over time. Control NB had diameters of 45±44 μm (FIG. 15, Panel A), 42±44 μm (FIG. 15, Panel B), and 24±25 μm (FIG. 15, Panel C) prior to LIPUS stimulation, from n=50 NB counted. Shear method 1 NB diameters could not be quantified due to the indiscernible proximity of the NB.

### 4. Characterization of NB Through TEM Imaging

TEM images of the final selected fabrication procedure, shearing method 1, and the 40 mg HSA control samples were taken for an undiluted version and a 10× diluted version.

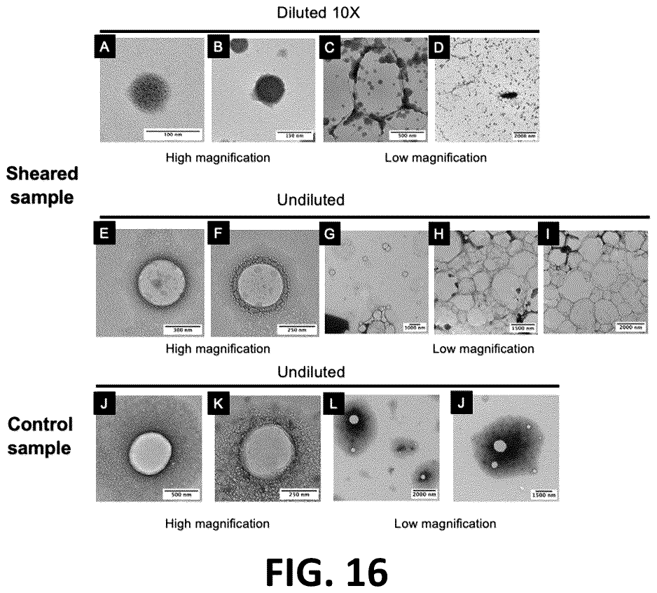

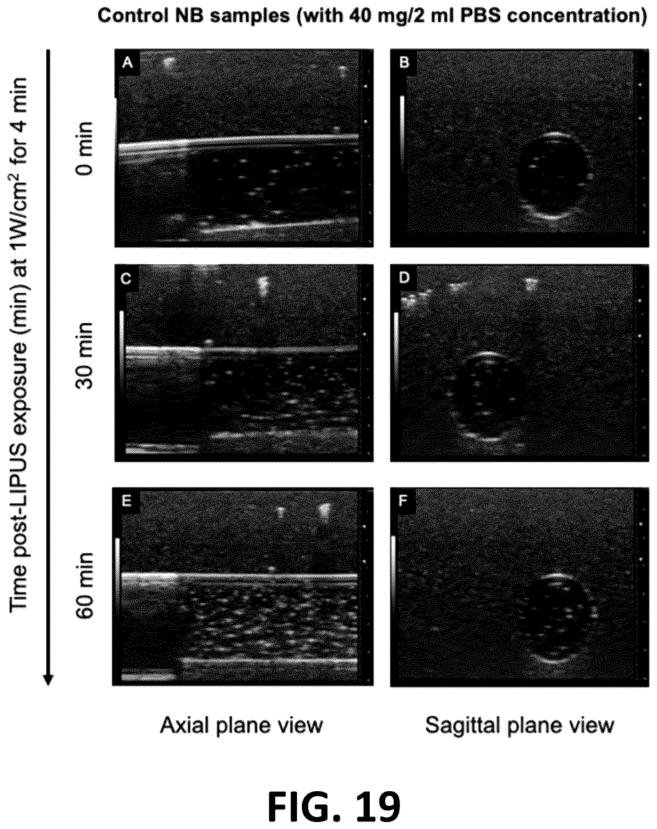

In FIG. 16, Panels A and B, the 10× diluted sheared NB had a diameter of approximately 70 nm and 130 nm, respectively. In FIG. 16, Panels C and D, quantification was more complex as there were multiple NB in lower magnification. A random sample size of n=30 NB was used for FIG. 16, Panel C and averaged the diameter to be approximately 100±25 nm; similarly, for FIG. 16, Panel D, the sample size was n=50 NB, and an average diameter of 127 nm was recorded. For FIG. 16, Panels E and F, it was recorded that the undiluted sheared NB had a diameter of 527 nm and 405 nm, respectively. In FIG. 16, Panel G, in a sample size of n=10, the average diameter was 427 nm with NB selected ranging from 290 to 500 nm. In FIG. 16, Panel H, in a sample size of n=10 NB, smaller white NB spheres were around 280 nm, while the larger white ones had diameters around 1000-1200 nm, and observed similar values for FIG. 16, Panel I. The black spheres were also quantified for FIG. 16, Panel H, with a sample size of n=11, had an average diameter of 177±110 nm. It is ambiguous as to the reason some spheres are white or black in color, which may be attributed to size; another possibility is that the black spheres are nanodroplets with higher density and the white spheres are NB with lower density due to their gas core. There appears to be a size difference between the undiluted and diluted 10× NB samples, with the diluted NB having smaller diameters. Increasing dilution ratios have been observed to reduce NB size in a nanoparticle tracking analyzer (NTA), which may be attributed to more space for gas diffusion across the particle, thus reducing their size. Furthermore, the NB could aggregate together in the undiluted sample due to their higher concentration, which complicates the process of quantifying individually isolated NB. For the control sample (FIG. 16, Panels J and K), high magnification imaging revealed diameters of 804 nm and 434 nm, respectively. FIG. 19, Panel L had a sample size of n=10 NB with a total average diameter of 110 nm, with larger NB around 200-300 nm while the smaller ones are 40-70 nm in diameter. FIG. 19, Panel J corroborates this observation, with a sample size of n=6 NB, the largest NB in frame has a diameter of 1009 nm, while the rest range between 180-330 nm.

### 5. US Contrast Imaging of NB in a Phantom Agarose Vessel

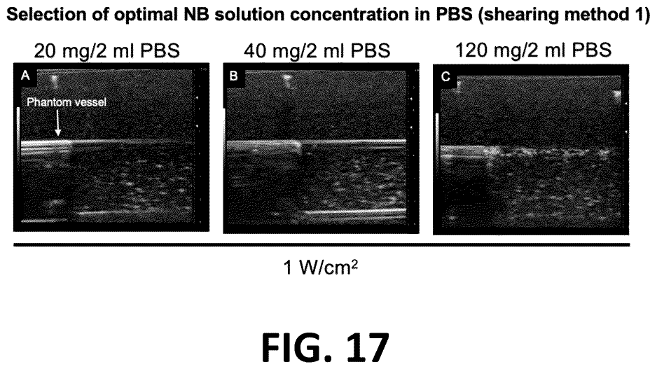

To investigate the potential of NB as a US contrast agent for diagnostic applications, shear method 1 and control NB were injected into a phantom vessel and isolated to the bottom ~2 inches of the vessel. To conduct larger imaging studies, the concentration of the optimal NB solution must be calculated through comparing intensities, while also considering a realistic concentration in 2 ml PBS. 20 mg/2 ml would be the typical concentration ratio (1×), 40 mg/2 ml was twice the original, and 120 mg/2 ml was 6× the original. Intensity comparisons were conducted as per the Methods, by selecting a ROI and a background for comparison. After intensity evaluations on ImageJ, 40 mg/2 ml (FIG. 17, Panel B) was the optimal NB solution concentration to proceed US imaging studies with, as it had a mean intensity value of 41.9, while the other 2 samples had intensities of 29.8 (20 mg/2 ml) and 38.8 (120 mg/2 ml).

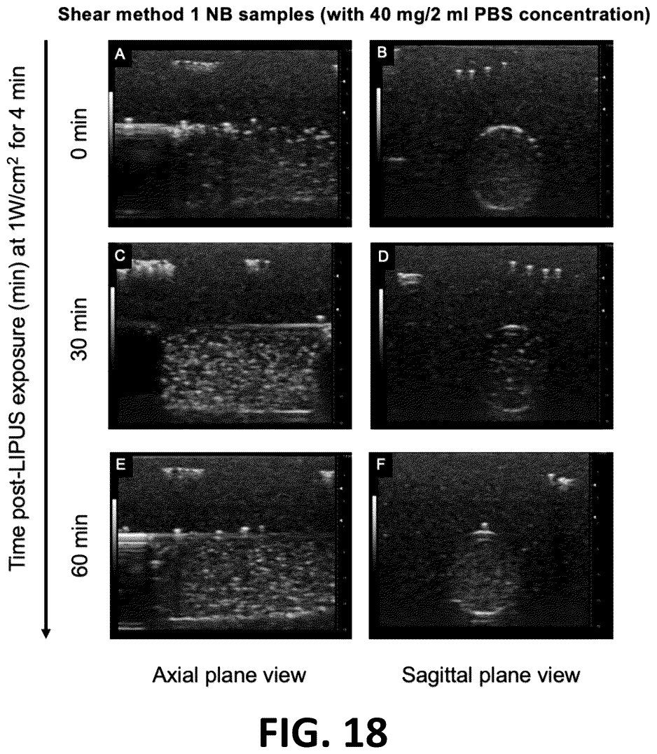

The shear method 1 and control NB samples were then imaged overtime, as seen in FIGS. 13-14 in NB microscope image studies, though the timespan was only up to 1 hour. Only images in axial view were evaluated for intensity. After comparing the same ROI and background overlays across FIG. 18, Panels A, C, and E, ROI intensity increased from 0 min (41.479) to 30 min (60.624) and remained consistent after 60 min (60.283). However, accounting for signal to noise ratio, it was concluded that the NB US contrast abilities were maximized at the 30 min timepoint, as its ratio was 4.81 compared to 3.02 and 3.55 for 0 min and 60 min timepoints, respectively. This reflects a more apt contrast and intensity efficiency as it accounts for background noise. Similarly for the control NB, an overall increase in intensity was observed in the ROI, transforming from 18.27 at 0 min to 11.16 at 30 min, and finally 36. 71 at 60 min, which is also the timepoint with the highest intensity and contrast when accounting for the signal to noise ratio. However, it still falls behind the higher intensity values seen in the shear method 1 NB samples in FIG. 18 and Table 1. Additionally, results were not consistent across the n=5 sample size in the control NB study, with some images extremely bright and others too dim. This could be attributed to inconsistency in concentration from measuring the liquid-state pellet after synthesis and centrifugation, as this can result in an uneven distribution of NB in the solution for phantom vessel experiments.

TABLE 1
Grayscale intensity values of 40 mg/2
ml solution of sheared NBs over time

| | ROI | Background | Signal to |
|---|---|---|---|
| Timepoint | intensity | Intensity | noise ratio |
| 0 min | 41.479 | 13.697 | 3.02832737 |
| 30 min | 60.624 | 12.588 | 4.81601525 |
| 60 min | 60.283 | 16.938 | 3.55903885 |

Grayscale intensity values of US shear method 1 NB over time.
n = 1 analysis from FIG. 18, Panels A, C, and E

TABLE 2
Grayscale intensity values of 40 mg/2
ml solution of control NBs over time

| | ROI | Background | Signal to |
|---|---|---|---|
| Timepoint | intensity | Intensity | noise ratio |
| 0 min | 10.08 | 18.27 | 0.55172414 |
| 30 min | 19.22 | 11.16 | 1.72222222 |
| 60 min | 36.71 | 15.72 | 2.33524173 |

Grayscale intensity values of control NB over time.
n = 1 analysis from FIG. 19, Panels A, C, and E

### 6. Pilot US Imaging of Non-Sheared NB Injected In Vivo

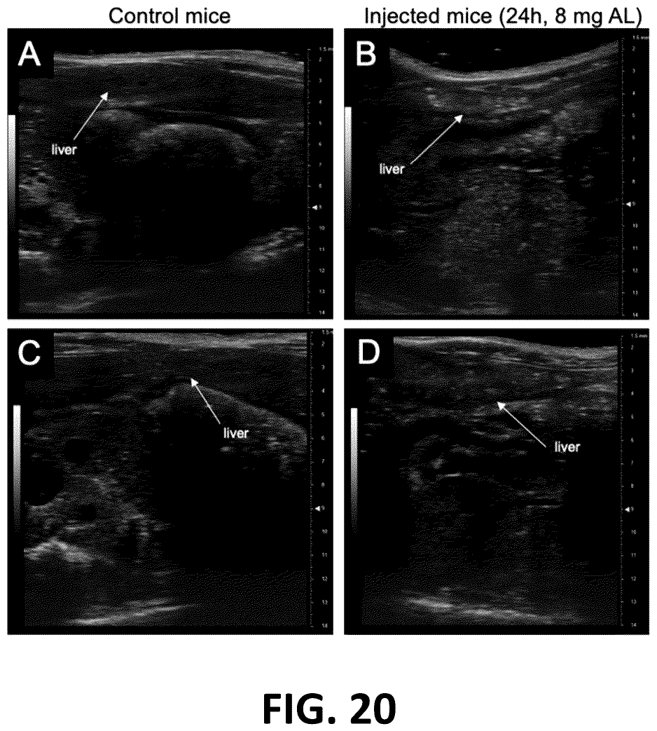

In the pilot study (as part of a separate, relevant project), control mice were evaluated alongside mice injected with 40 mg HSA concentration NB functionalized with 8 mg AL, which localizes to bone (FIG. 20). This evaluation took place 24 hours after the injection in the injected mice group. The mice were X-rayed to evaluate localized or regional fluorescence locations in organs or bone. The US imaging shows that NB are also extravasating and localizing into the liver, which is depicted as just beneath the skin in US. The mean intensity values for control are 25.68 (FIG. 18, Panel A) and 24.34 (FIG. 18, Panel C), as opposed to injected mice with intensity values of 46.58 (FIG. 18, Panel B) and 26.92 (FIG. 18, Panel D).

### CONCLUSION

In summation, a novel US shearing-based fabrication method for the synthesis of NB has been developed, which serves as a platform for multimodal applications in both drug/gene delivery systems and diagnostic US imaging uses. The NB-stabilizing albumin shell amount was first optimized, and then optimized further using a US-sheared method with a vertical slotted head stator setup; both characterizations and optimizations improved upon metrics such as monodispersity, effective diameter, and protein release over time. The sheared NB samples were also successfully imaged on an US machine using a phantom vessel post-LIPUS stimulation. Average grayscale intensity values also increased over a 1-hour timespan, sustaining approximately the same mean value at 30 min and 1 hour. This Example also demonstrated localization of NB in vivo into the liver, with increased intensity in the injected mice liver. The compositions and methods of the present disclosure may be utilized in treating bone in osteoporosis, which currently has no cure or FDA-approved treatments for CTSK silencing and downregulation. In addition, the compositions and methods of the present disclosure provide for the incorporation of CTSK siRNA and the bone-localizing biomarker, AL, into the NB. The present disclosure also has strong commercial potential, given its increased synthesis volume and utility in large scale manufacturing. Ultimately, the novel US-shearing based fabrication method for NB synthesis not only has applications in US contrast imaging and treating osteoporosis, but it also provides a platform for programmable, personalized medicine and delivery systems.

### Non-Illustrative Embodiments of the Inventive Concepts

Illustrative Embodiment 1. A method of preparing a substantially monodisperse, ultrasound responsive targeted nanobubble composition, the method comprising the steps of: (1) mixing at least one therapeutic agent with at least one gas and at least one polymer to form a first mixture; (2) sonicating and shearing the first mixture to form nanobubbles that comprise a polymer shell formed about a hollow core containing the at least one therapeutic agent and the at least one gas; (3) isolating the nanobubbles; and (4) contacting the nanobubbles with at least one targeting agent to form a second mixture and incubating the second mixture under conditions that allow the at least one targeting agent to be incorporated in the polymer shell and/or attached to a surface of the polymer shell to form the nanobubble composition; and wherein the nanobubble composition has a polydispersity index value in a range of from about 0.0 to about 0.20.

Illustrative Embodiment 2. The method of Illustrative Embodiment 1, wherein the nanobubbles have a diameter in a range of from about 20 nm to about 200 nm.

Illustrative Embodiment 3. The method of Illustrative Embodiment 1 or 2, wherein the shearing in step (2) is performed using a rotor-stator system.

Illustrative Embodiment 4. The method of any one of Illustrative Embodiments 1-3, wherein the gas is perfluorocarbon, and the polymer is albumin and/or polyethylene glycol (PEG).

Illustrative Embodiment 5. The method of any one of Illustrative Embodiments 1-4, wherein at least one of: step (2) is performed at a temperature in a range of from about 0° C. to about 4° C.; step (3) comprises a centrifugation step; and/or step (4) is performed at a temperature in a range of from about 0° C. to about 4° C. for a period in a range of from about 24 hours to about 48 hours.

Illustrative Embodiment 6. The method of any one of Illustrative Embodiments 1-5, wherein at least one imaging agent is added to step (1) and/or (4) so that the at least one imaging agent is disposed in the hollow core and/or incorporated in the polymer shell.

Illustrative Embodiment 7. The method of any one of Illustrative Embodiments 1-6, wherein the at least one targeting agent targets the nanobubble composition to at least a portion of a musculoskeletal system of a patient.

Illustrative Embodiment 8. The method of any one of Illustrative Embodiments 1-7, wherein at least one of: (a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof; (b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof; (c) the at least one therapeutic agent comprises Cathepsin K (CTSK) siRNA, and wherein the at least one targeting agent comprises alendronate and/or a bone targeting peptide; and/or (d) the at least one therapeutic agent comprises CTSK siRNA and at least one gene sequence selected from BMP and VEGF.

Illustrative Embodiment 9. A substantially monodisperse nanobubble composition produced by the method of any one of Illustrative Embodiments 1-8.

Illustrative Embodiment 10. A substantially monodisperse, ultrasound responsive targeted nanobubble composition, comprising: a plurality of nanobubbles that each comprise: a hollow core containing at least one gas; a polymer shell encircling the hollow core; at least one therapeutic agent disposed in the hollow core and/or encapsulated within the polymer shell; and at least one targeting agent incorporated in the polymer shell and/or attached to a surface of the polymer shell; and wherein the nanobubble composition has a polydispersity index value in a range of from about 0.0 to about 0.20.

Illustrative Embodiment 11. The nanobubble composition of Illustrative Embodiment 10, wherein the nanobubbles have a diameter in a range of from about 20 nm to about 200 nm.

Illustrative Embodiment 12. The nanobubble composition of Illustrative Embodiment 10 or 11, wherein the at least one targeting agent targets the nanobubble composition to at least a portion of a musculoskeletal system of a patient.

Illustrative Embodiment 13. The nanobubble composition of any one of Illustrative Embodiments 10-12, wherein at least one of: (a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof; (b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof; (c) the at least one therapeutic agent comprises Cathepsin K (CTSK) siRNA, and wherein the at least one targeting agent comprises alendronate and/or a bone targeting peptide; and/or (d) the at least one therapeutic agent comprises CTSK siRNA and at least one gene sequence selected from BMP and VEGF.

Illustrative Embodiment 14. The nanobubble composition of any one of Illustrative Embodiments 10-13, wherein the at least one gas is perfluorocarbon gas, the polymer shell comprises albumin and/or PEG, and the at least one targeting agent comprises alendronate and/or at least one bone targeting peptide.

Illustrative Embodiment 15. The nanobubble composition of any one of Illustrative Embodiments 10-14, further comprising at least one imaging agent disposed in the hollow core and/or incorporated in the polymer shell.

Illustrative Embodiment 16. A library, comprising: a plurality of substantially monodisperse nanobubble compositions of any one of Illustrative Embodiments 9-15, wherein each nanobubble composition has a different size in a range of from about 20 nm to about 200 nm in diameter, and wherein each nanobubble composition has a polydispersity index value in a range of from about 0.0 to about 0.20.

Illustrative Embodiment 17. The library of Illustrative Embodiment 16, wherein the plurality of nanobubble compositions comprise a nanobubble composition having a size of about 25 nm in diameter, a nanobubble composition having a size of about 50 nm in diameter, a nanobubble composition having a size of about 100 nm in diameter, a nanobubble composition having a size of about 150 nm in diameter, and a nanobubble composition having a size of about 200 nm in diameter.

Illustrative Embodiment 18. A kit or system, comprising at least one nanobubble composition of any one of Illustrative Embodiments 9-15 or at least one library of Illustrative Embodiments 16 or 17.

Illustrative Embodiment 18A. The kit or system of Illustrative Embodiment 18, further defined as comprising at least two nanobubble compositions, at least three nanobubble compositions, at least four nanobubble compositions, or at least five nanobubble compositions.

Illustrative Embodiment 19. A method, comprising the step of: (1) administering an effective amount of the library of Illustrative Embodiment 16 or 17 to a patient in need thereof.

Illustrative Embodiment 19A. The method of Illustrative Embodiment 19, wherein at least two of the different sized substantially monodisperse nanobubble compositions rupture at different times and thus deliver the at least one therapeutic agent in intervals over an extended period of time when compared to the rupture of substantially monodisperse nanobubble compositions that have the same size.

Illustrative Embodiment 20. A method, comprising the step of: (1) administering an effective amount of at least one ultrasound responsive targeted nanobubble composition to a patient in need thereof, wherein the nanobubble composition comprises a hollow core containing at least one gas, a polymer shell encircling the hollow core, at least one therapeutic agent disposed in the hollow core and/or encapsulated within the polymer shell, and at least one targeting agent incorporated in the polymer shell and/or attached to a surface of the polymer shell, wherein the at least one targeting agent targets the nanobubble composition to at least a portion of a musculoskeletal system of the patient, and wherein the nanobubble composition has a polydispersity index value in a range of from about 0.0 to about 0.20.

Illustrative Embodiment 21. The method of Illustrative Embodiment 20, wherein the nanobubble composition has a diameter in a range of from about 20 nm to about 200 nm.

Illustrative Embodiment 22. The method of Illustrative Embodiment 20 or 21, further comprising the steps of: (2) allowing the nanobubble composition to travel through the patient so that the targeting agent binds to a target within at least a portion of the musculoskeletal system of the patient; and (3) exposing the patient to ultrasound.

Illustrative Embodiment 23. The method of Illustrative Embodiment 22, wherein the ultrasound is selected from the group consisting of low intensity pulsed ultrasound (LIPUS), low intensity continuous ultrasound (LICUS), and focused ultrasound (FUS).

Illustrative Embodiment 24. The method of Illustrative Embodiment 22 or 23, wherein the patient is exposed to ultrasound in step (3) for in vivo diagnostic imaging of the target within the musculoskeletal system of the patient.

Illustrative Embodiment 25. The method of any one of Illustrative Embodiments 22-24, wherein in step (3), the ultrasound emits acoustic waves based on a determined frequency with an intensity and duration to rupture the nanobubble composition at the target within the patient.

Illustrative Embodiment 26. The method of Illustrative Embodiment 25, further comprising the step of: (4) administering an effective amount of a second ultrasound responsive targeted nanobubble composition to a patient in need thereof, wherein the nanobubble composition comprises a hollow core containing at least one gas, at least one therapeutic agent disposed in the hollow core, a polymer shell encircling the hollow core, and at least one targeting agent incorporated in the polymer shell, wherein the at least one targeting agent targets the nanobubble composition to at least a portion of a musculoskeletal system of the patient.

Illustrative Embodiment 27. The method of Illustrative Embodiment 26, wherein in the nanobubble composition of step (1), the at least one therapeutic agent comprises Cathepsin K (CTSK) siRNA, and the targeting agent comprises alendronate and/or at least one bone targeting peptide, and wherein in the nanobubble composition of step (4), the at least one therapeutic agent comprises at least one gene sequence selected from BMP and VEGF.

Illustrative Embodiment 28. The method of any one of Illustrative Embodiments 20-27, further comprising the step of exposing the patient to ultrasound prior to performing step (1).

Illustrative Embodiment 29. The method of any one of Illustrative Embodiments 20-28, wherein at least one of: (a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof; (b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof; (c) the at least one therapeutic agent comprises Cathepsin K (CTSK) siRNA, and wherein the at least one targeting agent comprises alendronate and/or at least one bone targeting peptide; and/or (d) the at least one therapeutic agent comprises CTSK siRNA and at least one gene sequence selected from BMP and VEGF.

Illustrative Embodiment 30. The method of any one of Illustrative Embodiments 20-29, wherein the gas is perfluorocarbon, and the polymer is albumin and/or PEG.

Illustrative Embodiment 31. The method of any one of Illustrative Embodiments 20-30, further defined as a method of treating a bone condition, disease, or disorder in the patient.

Illustrative Embodiment 32. The method of Illustrative Embodiment 31, wherein the bone condition, disease, or disorder comprises osteoporosis.

Illustrative Embodiment 33. The method of Illustrative Embodiment 31 or 32, wherein the bone condition, disease, or disorder is selected from the group consisting of a bone fracture, a bone defect, osteoarthritis, and a cancer.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

What is claimed is:

1. A method of preparing a substantially monodisperse, ultrasound responsive targeted nanobubble composition, the method comprising the steps of:
(1) mixing at least one therapeutic agent with at least one gas and at least one polymer to form a first mixture;
(2) sonicating and shearing the first mixture to form nanobubbles that comprise a polymer shell formed about a hollow core containing the at least one therapeutic agent and the at least one gas;
(3) isolating the nanobubbles; and
(4) contacting the nanobubbles with at least one targeting agent to form a second mixture and incubating the second mixture under conditions that allow the at least one targeting agent to be incorporated in the polymer shell and/or attached to a surface of the polymer shell to form the nanobubble composition; and
wherein the nanobubble composition has a polydispersity index value in a range of from about 0.0 to about 0.20.

2. The method of claim 1, wherein the nanobubbles have a diameter in a range of from about 20 nm to about 200 nm.

3. The method of claim 1, wherein the shearing in step (2) is performed using a rotor-stator system.

4. The method of claim 1, wherein the gas is perfluorocarbon, and the polymer is albumin and/or polyethylene glycol (PEG).

5. The method of claim 1, wherein at least one of:
step (2) is performed at a temperature in a range of from about 0° C. to about 4° C.;
step (3) comprises a centrifugation step; and/or
step (4) is performed at a temperature in a range of from about 0° C. to about 4° C. for a period in a range of from about 24 hours to about 48 hours.

6. The method of claim 1, wherein at least one imaging agent is added to step (1) and/or (4) so that the at least one imaging agent is disposed in the hollow core and/or incorporated in the polymer shell.

7. The method of claim 1, wherein the at least one targeting agent targets the nanobubble composition to at least a portion of a musculoskeletal system of a patient.

8. The method of claim 1, wherein at least one of:
(a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof;
(b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof;
(c) the at least one therapeutic agent comprises Cathepsin K (CTSK) siRNA, and wherein the at least one targeting agent comprises alendronate and/or at least one bone targeting peptide; and/or
(d) the at least one therapeutic agent comprises CTSK siRNA and at least one gene sequence selected from BMP and VEGF.

9. A substantially monodisperse nanobubble composition produced by the method of any one of claims 1-8.

10. A substantially monodisperse, ultrasound responsive targeted nanobubble composition, comprising:
a plurality of nanobubbles that each comprise:
a hollow core containing at least one gas;
a polymer shell encircling the hollow core;
at least one therapeutic agent disposed in the hollow core and/or encapsulated within the polymer shell; and
at least one targeting agent incorporated in the polymer shell and/or attached to a surface of the polymer shell; and
wherein the nanobubble composition has a polydispersity index value in a range of from about 0.0 to about 0.20.

11. The nanobubble composition of claim 10, wherein the nanobubbles have a diameter in a range of from about 20 nm to about 200 nm.

12. The nanobubble composition of claim 10, wherein the at least one targeting agent targets the nanobubble composition to at least a portion of a musculoskeletal system of a patient.

13. The nanobubble composition of claim 10, wherein at least one of:
(a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof;
(b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof;
(c) the at least one therapeutic agent comprises Cathepsin K (CTSK) siRNA, and wherein the at least one targeting agent comprises alendronate and/or at least one bone targeting peptide; and/or
(d) the at least one therapeutic agent comprises CTSK siRNA and at least one gene sequence selected from BMP and VEGF.

14. The nanobubble composition of claim 10, wherein the at least one gas is perfluorocarbon gas, the polymer shell comprises albumin and/or PEG, and the at least one targeting agent comprises alendronate and/or at least one bone targeting peptide.

15. The nanobubble composition of claim 10, further comprising at least one imaging agent disposed in the hollow core and/or incorporated in the polymer shell.

16. A library, comprising:
a plurality of substantially monodisperse nanobubble compositions of any one of claims 9-15, wherein each nanobubble composition has a different size in a range of from about 20 nm to about 200 nm in diameter, and wherein each nanobubble composition has a polydispersity index value in a range of from about 0.0 to about 0.20.

17. The library of claim 16, wherein the plurality of nanobubble compositions comprise a nanobubble composition having a size of about 25 nm in diameter, a nanobubble composition having a size of about 50 nm in diameter, a nanobubble composition having a size of about 100 nm in diameter, a nanobubble composition having a size of about 150 nm in diameter, and a nanobubble composition having a size of about 200 nm in diameter.

18. A kit, comprising at least one nanobubble composition of any one of claims 9-15 or at least one library of claim 16 or 17.

19. A method, comprising the step of:
(1) administering an effective amount of the library of claim 16 or 17 to a patient in need thereof.

20. A method, comprising the step of:
(1) administering an effective amount of at least one ultrasound responsive targeted nanobubble composition to a patient in need thereof, wherein the nanobubble composition comprises a hollow core containing at least one gas, a polymer shell encircling the hollow core, at least one therapeutic agent disposed in the hollow core and/or encapsulated within the polymer shell, and at least one targeting agent incorporated in the polymer shell and/or attached to a surface of the polymer shell, wherein the at least one targeting agent targets the nanobubble composition to at least a portion of a musculoskeletal system of the patient, and wherein the nanobubble composition has a polydispersity index value in a range of from about 0.0 to about 0.20.

21. The method of claim 20, wherein the nanobubble composition has a diameter in a range of from about 20 nm to about 200 nm.

22. The method of claim 20, further comprising the steps of:
(2) allowing the nanobubble composition to travel through the patient so that the targeting agent binds to a target within at least a portion of the musculoskeletal system of the patient; and
(3) exposing the patient to ultrasound.

23. The method of claim 22, wherein the ultrasound is selected from the group consisting of low intensity pulsed ultrasound (LIPUS), low intensity continuous ultrasound (LICUS), and focused ultrasound (FUS).

24. The method of claim 22, wherein the patient is exposed to ultrasound in step (3) for in vivo diagnostic imaging of the target within the musculoskeletal system of the patient.

25. The method of claim 22, wherein in step (3), the ultrasound emits acoustic waves based on a determined frequency with an intensity and duration to rupture the nanobubble composition at the target within the patient.

26. The method of claim 25, further comprising the step of:
(4) administering an effective amount of a second ultrasound responsive targeted nanobubble composition to a patient in need thereof, wherein the nanobubble composition comprises a hollow core containing at least one gas, at least one therapeutic agent disposed in the hollow core, a polymer shell encircling the hollow core, and at least one targeting agent incorporated in the polymer shell, wherein the at least one targeting agent targets the nanobubble composition to at least a portion of a musculoskeletal system of the patient.

27. The method of claim 26, wherein in the nanobubble composition of step (1), the at least one therapeutic agent comprises Cathepsin K (CTSK) siRNA, and the targeting agent comprises alendronate and/or at least one bone targeting peptide, and wherein in the nanobubble composition of step (4), the at least one therapeutic agent comprises at least one gene sequence selected from BMP and VEGF.

28. The method of claim 20, further comprising the step of exposing the patient to ultrasound prior to performing step (1).

29. The method of claim 20, wherein at least one of:
(a) the at least one targeting agent comprises an siRNA selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin (SOST) siRNA, receptor activator of nuclear factor kappa-B ligand (RANKL) siRNA, multi-siRNA, and combinations thereof;
(b) the at least one therapeutic agent comprises at least one gene sequence selected from the group consisting of vascular endothelial growth factor (VEGF), bone morphogenetic proteins (BMP), hepatocyte growth factor (HGF), osteocalcin, and combinations thereof;
(c) the at least one therapeutic agent comprises Cathepsin K (CTSK) siRNA, and wherein the at least one targeting agent comprises alendronate and/or at least one bone targeting peptide; and/or
(d) the at least one therapeutic agent comprises CTSK siRNA and at least one gene sequence selected from BMP and VEGF.

30. The method of claim 20, wherein the gas is perfluorocarbon, and the polymer is albumin and/or PEG.

31. The method of claim 22, further defined as a method of treating a bone condition, disease, or disorder in the patient.

32. The method of claim 31, wherein the bone condition, disease, or disorder comprises osteoporosis.

33. The method of claim 31, wherein the bone condition, disease, or disorder is selected from the group consisting of a bone fracture, a bone defect, osteoarthritis, and a cancer.
